(19) United States
(12) Patent Application Publication
(10) Pub. No.: US 2026/0335397 A1
(43) Pub. Date: Oct. 29, 2026

(54) TREATMENT OF GEOGRAPHIC ATROPHY

(71) Applicant: Apellis Pharmaceuticals, Inc., Waltham, MA (US)

(72) Inventors: Cedric Francois, Prospect, KY (US); Pascal Deschatelets, West Newton, MA (US); Federico Grossi, Needham, MA (US)

(21) Appl. No.: 19/115,645

(22) PCT Filed: Sep. 29, 2023
(86) PCT No.: PCT/US2023/034207

§ 371 Date: Mar. 26, 2025

Related U.S. Application Data
(60) Provisional application No. 63/412,334, filed on Sep. 30, 2022.
(60) Provisional application No. 63/461,177, filed on Apr. 21, 2023.
(60) Provisional application No. 63/581,908, filed on Sep. 11, 2023.

(51) Int. Cl. A61K 38/12; A61K 47/60; A61P 27/02

(52) U.S. Cl. CPC A61K 38/12; A61K 47/60; A61P 27/02

(57) ABSTRACT

Methods and compositions for treatment of geographic atrophy are described. A method of treating a subject suffering from or at risk of geographic atrophy (GA), e.g., GA secondary to age related macular degeneration (AMD), comprising intravitreally administering to an eye of the subject monthly or every other month a composition comprising pegcetacoplan, wherein: (a) the composition comprises about 15 mg/0.1 mL pegcetacoplan, and (b) visual function of the eye of the subject is maintained, or loss of visual function is reduced, relative to control. A control is level of visual function in a sham-treated subject (e.g., an untreated subject having the same level of risk and/or stage or severity of GA as the treated subject) or an average level of visual function in an untreated subject population.

### CROSS REFERENCE TO RELATED APPLICATIONS

The present application claims the benefit of U.S. Prov. Appln. No. 63/412,334 filed Sep. 30, 2022, U.S. Prov. Appln. No. 63/461,177 filed Apr. 21, 2023, and U.S. Prov. Appln. No. 63/581,908 filed Sep. 11, 2023, the contents of all of which are hereby incorporated by reference in their entireties.

### BACKGROUND

Geographic atrophy (GA) is an advanced form of age-related macular degeneration (AMD), a leading cause of blindness. GA lesions affect the central portion of the retina, known as the macula, which is responsible for central vision. GA is progressive and irreversible, leading to central visual impairment and permanent loss of vision. Based on published studies, approximately one million people have GA in the United States and five million people have GA globally (Rudnicka et al., Opththalmology 119:571-580 (2012); Wong et al., Lancet Glob Health 2: e106-116 (2014)). There are currently no approved treatments for GA.

### SUMMARY

In some aspects, the disclosure features a method of treating a subject suffering from or at risk of geographic atrophy (GA), e.g., GA secondary to age related macular degeneration (AMD), comprising intravitreally administering to an eye of the subject monthly (e.g., 30 days) or every other month (e.g., 60 days) a composition comprising pegcetacoplan, wherein: (a) the composition comprises about 15 mg/0.1 mL pegcetacoplan, and (b) visual function of the eye of the subject is maintained, or loss of visual function is reduced, relative to control. In some embodiments, a control is level of visual function in a sham-treated subject (e.g., an untreated subject having the same level of risk and/or stage or severity of GA as the treated subject) or an average level of visual function in an untreated subject population (e.g., an untreated subject population having the same level of risk and/or stage or severity of GA as the treated subject).

In some embodiments, visual function of the eye is assessed by microperimetry, monocular maximum reading speed, Functional Reading Independence (FRI) Index, best-corrected visual acuity (BCVA) (e.g., normal luminance BCVA (NL-BCVA) or low luminance BCVA (LL-BCVA)). In some embodiments, after the administration, level of visual function of the eye is about 5%, 10%, 20%, 30%, 40%, 50%, or 60% better relative to a control.

In some embodiments, visual function of the eye is assessed by microperimetry. In some embodiments, after the administration, mean threshold sensitivity of the eye is about 0.25, about 0.5, about 0.75, or about 1 db higher, relative to a control. In some embodiments, the eye of the subject has a geographic atrophy lesion, and microperimetry is assessed within an area peripheral to a lesion border. In some embodiments, microperimetry is assessed within an area within about 500 microns (e.g., within about 400 microns, within about 300 microns, within about 250 microns, within about 200 microns, within about 150 microns) of each side of the lesion border.

In some embodiments assessing visual function of the eye comprises assessing foveal light sensitivity. In some embodiments such assessment may comprise performing microperimetry at one or more points within the fovea. In some embodiments the subject retains foveal light sensitivity in an eye at the time treatment of that eye with pegcetacoplan is initiated. In some embodiments, after administration, retention of foveal light sensitivity is prolonged, relative to a control.

In some embodiments, visual function of the eye is assessed by BCVA. In some embodiments, after the administration, BCVA is improved by about 5%, 10%, 15%, 20%, 25% or more, relative to a control, e.g., a sham-treated subject (e.g., an untreated subject having the same level of risk and/or stage or severity of GA as the treated subject) or an average level of BCVA in an untreated subject population (e.g., an untreated subject population having the same level of risk and/or stage or severity of GA as the treated subject). In some embodiments, after the administration, BCVA is improved by about 1 letter, 2 letters, 3 letters, 4 letters, 5 letters, 6 letters, 8 letters, 10 letters or more, relative to a control, e.g., a sham-treated subject (e.g., an untreated subject having the same level of risk and/or stage or severity of GA as the treated subject) or an average level of BCVA in an untreated subject population (e.g., an untreated subject population having the same level of risk and/or stage or severity of GA as the treated subject). In some embodiments, BCVA is improved at about 6 months, about 12 months, about 18 months, about 24 months, about 36 months or more, after starting administration of pegcetacoplan. In some embodiments, after the administration, rate of decrease in BCVA score is reduced, relative to a control. In some embodiments, after about 6 months, about 12 months, about 18 months, about 24 months, about 36 months or more, of starting administration of pegcetacoplan, BCVA score reduction is about 5%, 10%, 15%, 20%, or 25% less than corresponding BCVA score reduction of a control, e.g., a sham-treated subject (e.g., an untreated subject having the same level of risk and/or stage or severity of GA as the treated subject) or an average level of BCVA in an untreated subject population (e.g., an untreated subject population having the same level of risk and/or stage or severity of GA as the treated subject). In some embodiments, the subject has a geographic atrophy lesion at a distance of about 200 μm, about 250 μm, about 300 μm or greater from foveal center of the eye.

In some embodiments, visual function of the eye is assessed by a visual function questionnaire. In some embodiments, after the administration, visual function questionnaire score is improved by about 5%, 10%, 15%, 20%, 25% or more, relative to a control, e.g., a sham-treated subject (e.g., an untreated subject having the same level of risk and/or stage or severity of GA as the treated subject) or an average visual function questionnaire score in an untreated subject population (e.g., an untreated subject population having the same level of risk and/or stage or severity of GA as the treated subject). In some embodiments, after the administration, visual function questionnaire score is improved by about 1 point, 2 points, 3 points, 4 points, 5 points, 6 points, 8 points, 10 points or more, relative to a control, e.g., a sham-treated subject (e.g., an untreated subject having the same level of risk and/or stage or severity of GA as the treated subject) or an average visual function questionnaire score in an untreated subject population (e.g., an untreated subject population having the same level of risk and/or stage or severity of GA as the treated subject). In some embodiments, visual function questionnaire score is improved at about 6 months, about 12 months, about 18 months, about 24 months, about 36 months or more, after starting administration of pegcetacoplan. In some embodiments, after the administration, rate of decrease in visual function questionnaire score is reduced, relative to a control. In some embodiments, after about 6 months, about 12 months, about 18 months, about 24 months, about 36 months or more, of starting administration of pegcetacoplan, visual function questionnaire score reduction is about 10%, 20%, 30%, 40%, 50%, 60%, 70%, or 80% less than corresponding visual function questionnaire score reduction of a control, e.g., a sham-treated subject (e.g., an untreated subject having the same level of risk and/or stage or severity of GA as the treated subject) or an average visual function questionnaire score in an untreated subject population (e.g., an untreated subject population having the same level of risk and/or stage or severity of GA as the treated subject). In some embodiments, the visual function questionnaire is VFQ-25. In some embodiments, the subject has a geographic atrophy lesion at a distance of about 200 μm, about 250 μm, or about 300 μm or more from foveal center of the eye.

In some aspects, the disclosure features a method of decreasing new scotomatous points in an eye of a subject diagnosed with geographic atrophy, the method comprising administering to the eye of the subject a composition comprising about 15 mg pegcetacoplan for at least 24 months, and wherein the number of new scotomatous points is decreased relative to control. In some embodiments, development of new scotomatous points within a distance of about 250 microns from a GA lesion border is reduced relative to a control.

In some aspects, described herein is a method of improving the quality of life of a subject diagnosed with geographic atrophy, the method comprising administering a composition comprising about 15 mg pegcetacoplan to an eye of the subject, wherein said eye has a geographic atrophy lesion greater than or equal to 250 μm away from the foveal center.

In some embodiments, the composition is administered for at least about 2 months, about 4 months, about 6 months, about 8 months, about 10 months, about 12 months, about 14 months, about 16 months, about 18 months, about 20 months, about 22 months, about 24 months, about 30 months, about 36 months, about 42 months, or longer.

In some embodiments, the composition comprises about 5% to about 6% trehalose.

In some embodiments, the composition is administered monthly (e.g., 30 days). In some embodiments, the composition is administered every other month (e.g., 60 days).

In some embodiments, the method further comprises administering an anti-VEGF agent to the subject (e.g., intravitreally administering an anti-VEGF agent to the same eye of the subject to which the composition was administered).

In some embodiments, the method further comprises diagnosing the subject as suffering from or at risk of geographic atrophy (GA), e.g., GA secondary to age related macular degeneration (AMD). In some embodiments, the diagnosing step comprises fundus autofluorescence (FAF) and/or optical coherence tomography (OCT). In some embodiments, the method further comprises selecting the subject for treatment if total GA area is ≥2.5 mm² (e.g., ≥2.5 mm² and ≤17.5 mm²).

### BRIEF DESCRIPTION OF THE DRAWING

FIG. 1 shows the structure of pegcetacoplan (“APL-2”), assuming n of about 800 to about 1100 and a PEG of about 40 kD.

FIG. 2 is a schematic of the DERBY and OAKS Phase 3 study design of intravitreal pegcetacoplan in patients with GA.

FIG. 3 shows LS mean change (±SE) from baseline NL-BCVA Score (ETDRS Letters) from the DERBY and OAKS studies combined. Least square (LS) means estimated from a mixed effects model for repeated measures (MMRM). The mITT population was used for the analysis, defined as all randomized patients who received at least 1 injection of pegcetacoplan or sham and have baseline and at least one post baseline value of GA lesion area in the study eye. BCVA=best-corrected visual acuity; ETDRS=Early Treatment of Diabetic Retinopathy Study; GA=geographic atrophy; LS=least square; M=month; mITT=modified intent-to-treat; MMRM=mixed-effects model for repeated measures; NL=normal luminance; PEOM=pegcetacoplan every other month; PM=pegcetacoplan monthly; SE=standard error.

FIG. 4 shows LS mean change (±SE) from baseline in mean threshold sensitivity of all points (dB) from microperimetry assessment of patients in the OAKS study. LS means estimated from MMRM analysis. Subjects in the mITT population who had a baseline value and at least one post-baseline value for mean threshold sensitivity or number of scotomatous points were included in the analysis. dB=decibel; LS=least square; M=month; mITT=modified intent-to-treat; MMRM=mixed-effects model for repeated measures; PEOM=pegcetacoplan every other month; PM=pegcetacoplan monthly; SE=standard error.

FIG. 5 shows a representative image of microperimetry analysis of perilesional area (area within 250 microns of each side of GA lesion border).

FIG. 6 shows LS mean change (±SE) from baseline in mean threshold sensitivity of perilesional points (dB) from post hoc microperimetry assessment of patients in the OAKS study. LS means estimated from MMRM analysis. Subjects in the mITT population who had a baseline value and at least one post-baseline value for perilesional mean threshold sensitivity or perilesional number of scotomatous points were included in the analysis. Perilesional area defined as −250 μm inside baseline atrophy border to +250 μm outside atrophy border. dB=decibel; LS=least square; M=month; mITT=modified intent-to-treat; MMRM=mixed-effects model for repeated measures; PEOM=pegcetacoplan every other month; PM=pegcetacoplan monthly; SE=standard error.

FIG. 7 shows LS mean change (±SE) from baseline in area of PR or RPE loss per total FOV (mm²) in study eye from OAKS study at month 12. LS means estimated from MMRM analysis. The mITT population was used for the analysis, defined as all randomized patients who received at least 1 injection of pegcetacoplan or sham and had baseline and at least one post-baseline value of GA lesion area in the study eye. FOV=field of view; LS=least square; M=month; mITT=modified intent-to-treat; MMRM=mixed-effects model for repeated measures; PEOM=pegcetacoplan every other month; PM=pegcetacoplan monthly; PR=photoreceptor; RPE=retinal pigment epithelium; SE=standard error.

FIG. 8 shows LS mean change (±SE) from baseline in area of PR or RPE loss per total FOV (mm²) in study eye from DERBY study at month 12. LS means estimated from MMRM analysis. The mITT population was used for the analysis, defined as all randomized patients who received at least 1 injection of pegcetacoplan or sham and had baseline and at least one post-baseline value of GA lesion area in the study eye. FOV-field of view; LS=least square; M=month; mITT=modified intent-to-treat; MMRM=mixed-effects model for repeated measures; PEOM=pegcetacoplan every other month; PM=pegcetacoplan monthly; PR=photoreceptor; RPE=retinal pigment epithelium; SE=standard error.

FIG. 9A is a schematic depicting central fovea and area assessed for “foveal occupancy”.

FIG. 9B shows baseline BCVA from OAKS and DERBY patients having increased levels of foveal occupancy. Analysis included patients with OCT images from SPECTRALIS machines (around 75% of OAKS and DERBY sample).

FIG. 10A shows BCVA change from baseline to month 24 for pegcetacoplan and sham treated OAKS and DERBY patients having lesions ≥250 μm away from the foveal center. “*” indicates adjusted difference, mean (SE).

FIG. 10B shows VFQ-25 composite baseline scores and composite change from baseline to month 24 for pegcetacoplan and sham treated OAKS and DERBY patients having lesions ≥250 μm away from the foveal center.

FIG. 11A shows BCVA change from baseline to month 24 for pegcetacoplan and sham treated OAKS and DERBY patients having lesions <250 μm away from the foveal center. “*” indicates adjusted difference, mean (SE).

FIG. 11B shows VFQ-25 composite baseline scores and composite change from baseline to month 24 for pegcetacoplan and sham treated OAKS and DERBY patients having lesions <250 μm away from the foveal center.

### DETAILED DESCRIPTION OF CERTAIN EMBODIMENTS

### I. Definitions

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the disclosure the antibody is a fragment such as a Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16:535-539. The antibody may be partially or completely humanized. An antibody may be polyclonal or monoclonal, though for purposes of the present disclosure monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, the term “about X” includes the number “X” and numbers that fall within a range of 5%, 10%, 15%, or 20% in either direction (greater than or less than) of the number X.

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered followed by one or more additional agents. In the separate administration protocol, two or more agents may be administered a few minutes apart, or a few hours apart, or a few days apart, or a few weeks apart. In some embodiments, two or more agents may be administered 1-2 weeks apart. In some embodiments, if two or more agents useful for treating the same disease are administered in combination, each of the two or more agents may be administered using a dosing regimen that would be used if such agent were being used as the sole agent for treating the disease.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the disclosure the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual or subject who is “suffering from” a disease, disorder, and/or condition has been diagnosed with and/or displays one or more symptoms of a disease, disorder, and/or condition.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect, such as an immune response to a viral vector described herein. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., GA, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered prophylactically, i.e., before development of any symptom or manifestation of the condition. Typically in this case the subject will be at risk of developing the condition.

### II. Complement System

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” were subsequently reversed in the scientific literature.

The alternative pathway is initiated by and amplified at, e.g., microbial surfaces and various complex polysaccharides. In this pathway, hydrolysis of C3 to C3(H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344(14): 1058-66, 2001.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b: C4b receptor), complement receptor type 2 (CR2), membrane cofactor protein (MCP; CD46), decay-accelerating factor (DAF, also known as CD55), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders.

### III. Compstatin Analogs

Methods of the disclosure include treatment of GA using compstatin analogs. Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3): 619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32(Pt 1): 28-32, 2004, Mallik, B., et al., J. Med. Chem., 274-286, 2005; Katragadda, M., et al. J. Med. Chem., 49:4616-4622, 2006; WO2007062249 (PCT/US2006/045539); WO2007044668 (PCT/US2006/039397), WO/2009/046198 (PCT/US2008/078593); WO/2010/127336 (PCT/US2010/033345) and discussion below.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure.

TABLE 1

| | | SEQ ID | Activity over |
|---|---|---|---|
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3xmore |
| Ac-V4Y/H9A | Ac-ICVYQDWGAHRCT-CONH2 | 10 | 14xmore |
| Ac-V4W/H9A-OH | Ac-ICVWQDWGAHRCT-COOH | 11 | 27xmore |
| Ac-V4W/H9A | Ac-ICVWQDWGAHRCT-CONH2 | 12 | 45xmore |
| Ac-V4W/H9A/T13dT-OH | Ac-ICVWQDWGAHRCdT-COOH | 13 | 55xmore |
| Ac-V4(2-Nal)/H9A | Ac-ICV(2-Nal)QDWGAHRCT-CONH2 | 14 | 99xmore |
| Ac V4(2-Nal)/H9A-OH | Ac-ICV(2-Nal)QDWGAHRCT-COOH | 15 | 38xmore |
| Ac V4(1-Nal)/H9A-OH | Ac-ICV(1-Nal)QDWGAHRCT-COOH | 16 | 30xmore |
| Ac-V42Igl/H9A | Ac-ICV(2-Igl)QDWGAHRCT-CONH2 | 17 | 39xmore |
| Ac-V42Igl/H9A-OH | Ac-ICV(2-Igl)QDWGAHRCT-COOH | 18 | 37xmore |
| Ac-V4Dht/H9A-OH | Ac-ICVDhtQDWGAHRCT-COOH | 19 | 5xmore |
| Ac-V4(Bpa)/H9A-OH | Ac-ICV(Bpa)QDWGAHRCT-COOH | 20 | 49xmore |
| Ac-V4(Bpa)/H9A | Ac-ICV(Bpa)QDWGAHRCT-CONH2 | 21 | 86xmore |
| Ac-V4(Bta)/H9A-OH | Ac-ICV(Bta)QDWGAHRCT-COOH | 22 | 65xmore |
| Ac-V4(Bta)/H9A | Ac-ICV(Bta)QDWGAHRCT-CONH2 | 23 | 64xmore |
| Ac-V4W/H9(2-Abu) | Ac-ICVWQDWG(2-Abu)HRCT-CONH2 | 24 | 64xmore |
| +G/V4W/H9A +AN-OH | H-GICVWQDWGAHRCTAN-COOH | 25 | 38xmore |
| Ac-V4(5fW)/H9A | Ac-ICV(5fW)QDWGAHRCT-CONH₂ | 26 | 31xmore |
| Ac-V4(5-MeW)/H9A | Ac-ICV(5-methyl-W)QDWGAHRCT-CONH₂ | 27 | 67xmore |
| Ac-V4(1-MeW)/H9A | Ac-ICV(1-methyl-W)QDWGAHRCT-CONH₂ | 28 | 264xmore |
| Ac-V4W/W7(5fW)/H9A | Ac-ICVWQD(5fW)GAHRCT-CONH₂ | 29 | 121xmore |
| Ac-V4(5fW)/W7(5fW)/H9A | Ac-ICV(5fW)QD(5fW)GAHRCT-CONH₂ | 30 | NA |
| Ac-V4(5-MeW)/W7(5fW)H9A | Ac-ICV(5-methyl-W)QD(5fW)GAHRCT- | 31 | NA |
| | CONH₂ | | |
| Ac-V4(1MeW)/W7(5fW)/H9A | Ac-ICV(1-methyl-W)QD(5fW)GAHRCT- | 32 | 264xmore |
| | CONH₂ | | |
| +G/V4(6fW)/W7(6fW)H9A+N- | H-GICV(6fW)QD(6fW)GAHRCTN-COOH | 33 | 126xmore |
| OH | | | |
| Ac-V4(1-formyl-W)/H9A | Ac-ICV(1-formyl-W)QDWGAHRCT-CONH₂ | 34 | 264xmore |
| Ac-V4(5-methoxy-W)/H9A | Ac-ICV(1-methyoxy-W)QDWGAHRCT- | 35 | 76xmore |
| | CONH₂ | | |
| G/V4(5f-W)/W7(5fW)/H9A+N- | H-GICV(5fW)QD(5fW)GAHRCTN-COOH | 36 | 112xmore |
| OH | | | |

NA = not available

In certain embodiments of the compositions and methods of the disclosure, the compstatin analog has a sequence selected from sequences 9-36. In some embodiments, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g., to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo (ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In some embodiments, the polymeric backbone or scaffold comprises multiple reactive functional groups, such as carboxylic acids, anhydride, or succinimide groups. The polymeric backbone or scaffold is reacted with the compstatin analogs. In some embodiments, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In some embodiments, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and for compounds depicted herein, a polyethylene glycol moiety is drawn with the oxygen atom on the right side of the repeating unit or the left side of the repeating unit.

In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

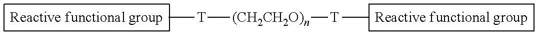

wherein each T and “Reactive functional group” is independently as defined below, and described in classes and subclasses herein, and n is as defined above and described in classes and subclasses herein.
Each T is independently a covalent bond or a C1-12 straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and each R^{x} is independently hydrogen or C1-6 aliphatic.
The Reactive functional group has the structure —COO—NHS.

Exemplary bifunctional PEGs of formula A include:

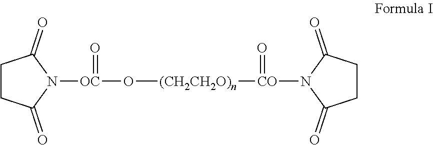

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

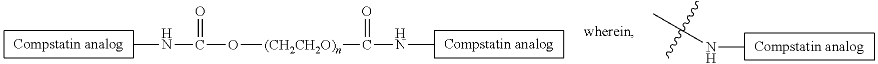

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

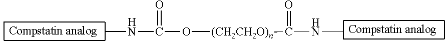

In some embodiments of formulae and embodiments described herein,

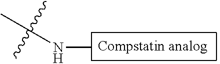 [SMILES: *NC]

represents point of attachment of a lysine side chain group in a compstatin analog having the structure:

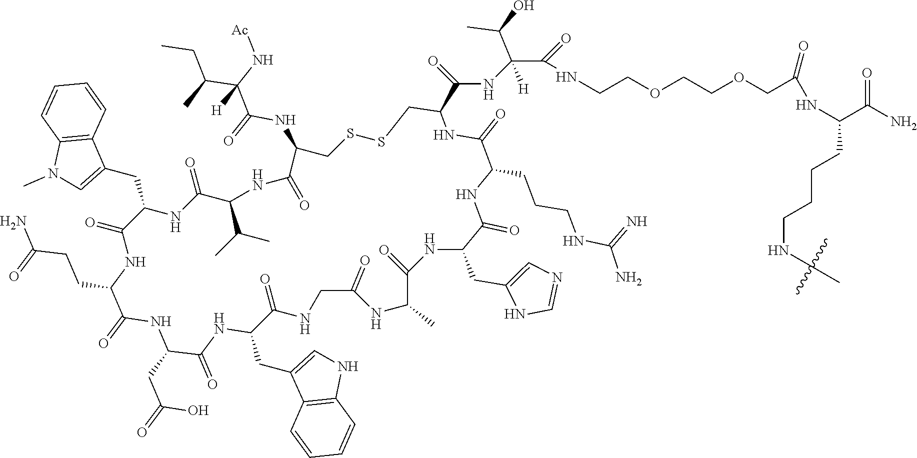 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)N1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)nCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O)OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the ε-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

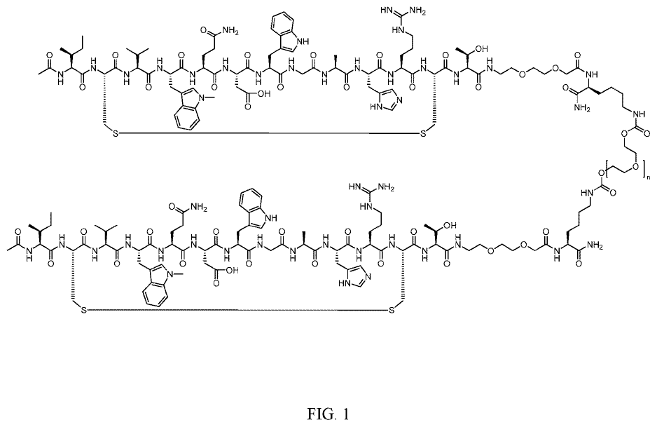

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 1. It will be understood that the PEG moiety may have a variety of different molecular weights or average molecular weights in various embodiments, as described herein. In certain embodiments, a compstatin analog is pegcetacoplan (“APL-2”), having the structure of the compound of FIG. 1 with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2->12)-(disulfide); or O,O′-bis[(S²,S¹²-cyclo {N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6,15}-carbonyl]polyethylene glycol (n=800-1100). Additional compstatin analogs are described in, e.g., WO 2012/155107 and WO 2014/078731.

### IV. Complement-Mediated Eye Disorders

In some embodiments, a compstatin analog described herein is introduced into the eye of a subject for treatment of an eye disorder such as age-related macular degeneration (AMD). For example, a compstatin analog may be introduced into the vitreous cavity (e.g., by intravitreal injection), for treatment of a subject suffering from or at risk of AMD. In some embodiments the AMD is neovascular (wet or exudative) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a compstatin analog as compared with control (e.g., patients given a sham injection). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD. In some embodiments, a subject has one eye with GA and one eye without GA. In some embodiments, a subject has one eye with GA and one eye without wet AMD. In some embodiments, a subject has one eye with GA and one eye with wet AMD. In some embodiments, a subject has one eye having both GA and neovascular AMD and one eye without wet AMD. In some embodiments, a subject has one eye having both GA and neovascular AMD and one eye with wet AMD. In some embodiments, both eyes of a subject have both GA and neovascular AMD.

Geographic atrophy (GA) is a late-stage disease manifestation in nonneovascular age-related macular degeneration (AMD) that progresses to severe central vision loss. Geographic atrophy has traditionally been defined on color fundus photography (CFP) as a sharply delineated circular or oval area of hypopigmentation or depigmentation in which choroidal vessels are visible. The size requirement for GA varies with the different studies, ranging from one eighth to one fourth of a disc area (corresponding roughly to 175 mm and 430 mm in diameter, respectively) on CFP (see, e.g., Bird et al., Surv. Ophthalmol. 39:367-374 (1995) and Schmitz-Valckenberg, Ophthalmologica 237:11-20 (2017)). Geographic atrophy was retained as a term for a late stage of AMD in the 2013 Beckman CFP classification of AMD (see Ferris et al., Ophthalmology 120:844-851 (2013)). As a clinical trial end point, the ability to slow GA expansion using a novel therapy has been approved by regulatory authorities, in which atrophy can be defined and quantified by fundus autofluorescence (FAF) (Csaky et al., Invest. Ophthalmol. Vis. Sci. 58:3456-3463 (2017); Holz et al., Ophthalmology 121:1079-1091 (2014); Schmitz-Valckenberg et al., Ophthalmology 123:361-368 (2016)). Recent trials aiming to slow progression of atrophy were required to enroll eyes with established regions of GA that could be measured reliably by a reading center, usually measuring at least 0.5 to 1.0 disc areas (1.25-2.5 mm²) (Cheng et al., Ophthalmol. Retina 2:518-525 (2018); Holz et al., JAMA Ophthalmol. 136:666-677 (2018); Rosenfeld et al., Ophthalmology 125:1556-1567 (2018)). In some embodiments, GA is assessed using FAF. In some embodiments, GA is assessed using optical coherence tomography (OCT).

Drusen are localized extracellular deposits of lipoproteinaceous material that accumulate between the retinal pigment epithelium (RPE) and the capillary network in the choroid (choriocapillaris), typically between the RPE and Bruch's membrane (a multilayered extracellular matrix complex that separates the RPE from the choriocapillaris). It will be appreciated that the term “druse” is sometimes used in the art to refer to a single such deposit (i.e., as a singular referent) while “drusen” is sometimes used in the art to refer to multiple such deposits (i.e., as a plural referent). As used herein, the term “drusen” should be understood to encompass a single “druse” or multiple “drusen” in various embodiments unless indicated to the contrary or clearly evident from the context. Also, reference to “a druse” should be understood to encompass reference to a single “druse” or multiple “drusen” in various embodiments unless indicated to the contrary or clearly evident from the context. Drusen are a clinical hallmark of AMD and are typically the earliest clinical finding in AMD. The presence, location, size, and number of drusen are factors used in the art for classifying AMD into stages and monitoring its progression. However, a few small drusen are commonly observed in the eyes of people over 40 years of age, most of whom do not go on to develop AMD. Drusen can be detected, assessed, and/or classified according to known methods, e.g., imaging, scanning laser ophthalmoscopy, and optical coherence tomography (OCT) (e.g., spectral domain optical coherence tomography (SD-OCT)). Additional methods are described in, e.g., WO2014/028861.

In some embodiments early, intermediate, or advanced AMD are defined in accordance with the classification scheme used in the Age-Related Eye Diseases Study (AREDS) (The Age-Related Eye Disease Study Research Group. A randomized, placebo-controlled, clinical trial of high-dose supplementation with vitamins C and E, beta carotene, and zinc for age-related macular degeneration and vision loss: AREDS report number 8. Arch Ophthalmol 2001; 119:1417-36). The classification of AMD from the AREDS is summarized as follows:

No AMD (AREDS category 1) is characterized by no or few small drusen (<63 microns in diameter).

Early AMD (AREDS category 2) is characterized by presence of a combination of multiple small drusen, few intermediate drusen (63 to 124 microns in diameter), or RPE abnormalities.

Intermediate AMD (AREDS category 3) is characterized by presence of extensive intermediate drusen, at least one large druse (at least 125 microns in diameter), or geographic atrophy not involving the center of the macula.

Advanced AMD (AREDS category 4) is characterized by geographic atrophy involving the center of the macula and/or neovascular macular degeneration. Neovascular macular degeneration is typically associated with manifestations of CNV and/or retinal or RPE detachment associated with subretinal serous fluid, exudates, and/or blood. Other manifestations of neovascular AMD may include retinal hard exudates, subretinal and sub-RPE fibrovascular proliferation, and/or disciform scar.

Other classification schemes, or modified forms of the AREDS scheme, may be used, such as the ICD10 classification system. The terms “extrafoveal” and “nonsubfoveal” lesions are used interchangeably herein, and refer to lesions having a distance from the atrophy junction to the center of the fovea of greater than zero. The terms “subfoveal” and “foveal” lesions are used interchangeably herein, and refer to lesions having a distance from the atrophy junction to the center of the fovea that is not greater than zero. In some embodiments, such distance is assessed using autofluorescence.

In some embodiments, age-related macular degeneration can be divided into 3 main clinical stages (early, intermediate, and late) based on overall disease severity (Ferris et al., Ophthalmology. 2013; 120:844-851), with decreased quality of life and significant visual impairment occurring during the late stage. Late AMD can be subdivided into the exudative form, which is characterized by the presence of macular neovascularization (MNV), and the nonexudative form, known as geographic atrophy (GA), which is characterized by progressive loss of macular photoreceptors, retinal pigment epithelium (RPE), and choriocapillaris (Holz et al., Ophthalmology. 2014; 121:1079-1091; Barbazetto et al., Arch Ophthalmol. 2003; 121:1253-1268). Geographic atrophy is also referred to as complete RPE and outer retinal atrophy (Holz et al., JAMA Ophthalmol. 2018; 136:666-677). The exudative form of AMD (eAMD) can be treated with intravitreal injections of vascular endothelial growth factor (VEGF) A inhibitors (Finger et al., BMC Ophthalmol. 2020; 20:294). Although anti-VEGF therapy has been shown to improve vision and reduce the risk of severe vision loss over several years in eyes with eAMD (Heier et al., Ophthalmology. 2012; 119:2537-2548; Rosenfeld et al., N Engl J Med. 2006; 355:1419-1431; Gillies et al., Ophthalmology. 2015; 122:1837-1845), the nonexudative form of AMD can continue to progress to macular atrophy, independent of the exudative process (Gune et al., Ophthalmology. 2020; 127:523-532). For most patients with late AMD, macular atrophy is the characteristic end stage of the disease process, resulting in progressive and irreversible loss of visual function as the area of macular atrophy enlarges. See also Wykoff et al., Ophthalmology 128:1325-1336, 2021.

### V. C5 Inhibitors

In some embodiments, a subject treated with a compstatin analog described herein, e.g., pegcetacoplan, has received one or more C5 inhibitors before treatment with the compstatin analog, receives one or more C5 inhibitors in combination with at least one dose of the compstatin analog, and/or continues to receive one or more C5 inhibitors during the entire treatment with the compstatin analog.

C5 inhibitors are known and/or commercially available. Non-limiting examples of C5 inhibitors include, e.g., eculizumab, ALXN1210 (ravulizumab), SKY59 (crovalimab), LFG316, REGN3918, ABP959, RA101495, Coversin, and ALNCC5 (described in, e.g., Risitano et al., Frontiers Immunology 10:1157 (2019)). Additional C5-targeting agents are described in, e.g., U.S. Pat. Nos. 9,718,880 and 9,079,949; and PCT Publs. WO2004106369; WO2010015608; WO2013093762; WO/2014/160129; WO2015134894; WO2015191951; WO/2016/040589; WO/2016/044419; WO2016098356; WO2016117346; WO2016123371; WO/2016/201301; WO2017104779; WO2017105939; WO2017212375; WO2017212391; WO/2017/214518; WO2017/217524; WO2017218515; WO2018106859; WO2018143266; WO2018165062; WO2018183449; WO2019014360; WO2019023564; WO2019084438; WO2019112984; WO2019118556; and WO2020006266.

In some embodiments, the C5 inhibitor is an anti-C5 antibody, e.g., an anti-C5 monoclonal antibody. In some embodiments, a C5 inhibitor is eculizumab or ravulizumab. In some embodiments, a C5 inhibitor is an antibody that binds to the same epitope as eculizumab or ravulizumab. In some embodiments, a C5 inhibitor is an antibody that competes for binding to C5 with eculizumab or ravulizumab. In some embodiments, a C5 inhibitor includes the same or substantially the same amino acid sequence as eculizumab or ravulizumab, or an antigen binding portion thereof.

### VI. Pharmaceutical Compositions and Administration

The disclosure provides and/or utilizes a variety of compositions comprising a compstatin analog. In various embodiments, a composition can have any feature or combination of features discussed herein so long as they are not mutually exclusive. The invention provides embodiments of such compositions, and methods of use thereof, in which the compstatin analog is any compstatin analog, e.g., an analog described herein.

Suitable preparations, e.g., substantially pure preparations of a compstatin analog or other active agent, may be combined with pharmaceutically acceptable carriers or vehicles, etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used in the compositions of this invention include, but are not limited to, water, physiological saline, Ringer's solution, sodium acetate or potassium acetate solution, 5% dextrose, and the like. The composition may include other components as appropriate for the formulation desired, e.g., as discussed herein. Supplementary active compounds, e.g., compounds independently useful for treating a subject suffering from a complement-mediated disorder, can also be incorporated into the compositions. The invention provides such pharmaceutical compositions comprising a compstatin analog and, optionally, a second active agent useful for treating a subject suffering from AMD.

In some embodiments, the invention provides a pharmaceutically-acceptable composition suitable for administration to humans, packaged together with a label approved by a government agency responsible for regulating pharmaceutical agents, e.g., the U.S. Food & Drug Administration. In some embodiments, the invention provides a pharmaceutical kit or pack for use in performing an intravitreal injection of a composition, e.g., pegcetacoplan, as described herein. In some embodiments, a pharmaceutical kit comprises one or more sterile needles. In some embodiments, a pharmaceutical kit comprises: (a) a transfer needle (e.g., a filtered transfer needle) and/or (b) a needle for injection. In some embodiments, the invention provides a pharmaceutical kit or pack comprising: (a) a single use vial comprising a pharmaceutical composition comprising pegcetacoplan, as described herein, (b) a transfer needle (e.g., a filtered transfer needle), and/or (c) a needle for injection. In some embodiments, a transfer needle and/or needle for injection are sterile needles. In some embodiments, a transfer needle and/or needle for injection are for administration of a single dose. In some embodiments, a transfer needle is a sterile 5-micron filter needle. In some embodiments, a needle for injection is a ½ inch, 29-gauge thin-wall injection needle. In some embodiments, a needle for injection is a ½ inch, 29-gauge thin-wall injection needle with a Luer-lock hub. In some embodiments, a transfer needle and/or needle for injection are contained in a sealed plastic tray comprising suitably sized compartments to house the transfer needle and/or needle for injection. In some embodiments, a pharmaceutical kit or pack further comprises a syringe for administration of a composition, e.g., pegcetacoplan, as described herein. In some embodiments, a syringe is a sterile 1-ml Luer-lock syringe. In some embodiments, a sterile 1-ml Luer-lock syringe has a 0.1 ml dose mark. In some embodiments instructions for administration are provided.

A pharmaceutical composition can be administered to a subject by any suitable route of administration including, but not limited to, intravitreally, intravenous, intramuscular, subcutaneously, by inhalation, by nasal delivery, intrathecally, intracranially, intraarterially, orally, rectally, transdermally, intradermally, subdermally, etc. It will be understood that “administration” encompasses directly administering a compound or composition to a subject, instructing a third party to administer a compound or composition to a subject, prescribing or suggesting a compound or composition to a subject, and, as appropriate, other means of making a compound or composition available to a subject.

Pharmaceutical compositions suitable for injectable use (e.g., intravitreal administration) typically include sterile aqueous solutions (where water soluble) or dispersions and sterile powders for the extemporaneous preparation of sterile injectable solutions or dispersion. Sterile solutions can be prepared by incorporating the compound in the required amount in an appropriate solvent, optionally with one or a combination of ingredients such as buffers such as acetates, citrates, lactates or phosphates; agents for the adjustment of tonicity; antibacterial agents such as benzyl alcohol or methyl parabens; antioxidants such as ascorbic acid, glutathione, or sodium bisulfite; chelating agents such as ethylenediaminetetraacetic acid; and other suitable ingredients etc., as desired, followed by filter-based sterilization. One of skill in the art will be aware of numerous physiologically acceptable compounds that may be included in a pharmaceutical composition. pH can be adjusted with acids or bases, such as hydrochloric acid or sodium hydroxide, if desired. Preferably solutions for injection are sterile and acceptably free of endotoxin. In some embodiments, solutions for injection are substantially free of preservative (e.g., are preservative free solutions). A composition is “substantially free” of a particular substance or substances if the composition contains no more than 0.1% weight/volume (w/v) of that substance or substances, e.g., no more than 0.01% w/v, e.g., no more than 0.001% w/v, of that substance or substances.

In certain embodiments a composition comprising a compstatin analog, or a composition to which a compstatin analog is to be added, comprises one or more non-reducing sugars or sugar alcohols. In certain embodiments a composition comprising a compstatin analog, or a composition to which a compstatin analog is to be added, comprises one or more reducing sugars, e.g., dextrose. One of ordinary skill in the art appreciates that a reducing sugar is a sugar that is capable of acting as a reducing agent, typically because it has a free aldehyde group or a free ketone group, e.g., when it assumes an open-chain form.

In some embodiments, compositions provided by the present disclosure include a compstatin analog and do not include a reducing sugar, e.g., dextrose, as a component. In some embodiments a composition comprising a compstatin analog, or to which a compstatin analog is to be added, is free or substantially free of one or more reducing sugars, e.g., dextrose. In certain embodiments a composition comprising a compstatin analog, or to which a compstatin analog is to be added, is free or substantially free of reducing sugars.

In some embodiments of any of the aspects described herein, a composition described herein comprises a compstatin analog described herein and water. In some embodiments of any of the aspects described herein, a composition described herein comprises a compstatin analog described herein, water, and one or both of a tonicity adjusting agent and a buffer substance. In some embodiments of any of the aspects described herein, a composition described herein consists essentially of or consists of a compstatin analog described herein and one or more specified components, e.g., a buffer substance and/or a tonicity adjusting agent. In some embodiments, such a composition consists of such substances in water as a pharmaceutically acceptable carrier. In some embodiments of any of the aspects described herein, a composition comprising a compstatin analog described herein has a pH as described herein.

In certain embodiments the composition comprises one or more excipients in an amount such that the composition is approximately isotonic with respect to normal human plasma. In some aspects, a composition is considered “isotonic with respect to normal human plasma” (also referred to simply as “isotonic”) if the concentration of solutes that cannot freely diffuse across a plasma membrane of a normal human cell, e.g., a normal human red blood cell or an epithelial cell, is between 260 mOsm/kg and 320 mOsm/kg, e.g., between 280 mOsm/kg and 300 mOsm/kg, e.g., 285 mOsm/kg and 295 mOsm/kg. In some embodiments a composition comprising a compstatin analog described herein and a pharmaceutically acceptable carrier has a pH of between 6.5 and 7.5, e.g., between 6.8 and 7.2, e.g., 7.0. In some embodiments, a composition comprising a compstatin analog and a pharmaceutically acceptable carrier has a pH of between 6.0 and 6.5, between 5.5 and 6.0, or between 5.0 and 5.5. In certain embodiments a yet lower pH, e.g., between 4.5 and 5.0 may be used. In particular embodiments, for example, a composition has a pH between 4.6 and 5.4, e.g., between 4.8 and 5.2, between 4.9 and 5.1, e.g., 5.0. In some embodiments the composition further comprises one or more pharmaceutically acceptable buffer substances appropriate to maintain the pH within a selected range (e.g., any of the afore-mentioned ranges). Suitable buffer substances are described herein (e.g., acetates, citrates, lactates or phosphates). In some embodiments the composition additionally or alternately comprises a salt, e.g., any of the pharmaceutically acceptable salts described herein.

In certain embodiments the buffer substance comprises sodium acetate. In certain embodiments the buffer substance, e.g., sodium acetate, is present at a concentration between 10 mM and 50 mM, e.g., 10 mM-15 mM, 15 mM-20 mM, 20 mM-25 mM, 25 mM-30 mM, 30 mM-35 mM, 35 mM-40 mM, 40 mM-50 mM. In particular embodiments the buffer substance is present at a concentration of about 15 mM, about 17.5 mM, about 20 mM, about 22.5 mM, about 25 mM, about 27.5 mM, or about 30 mM. In certain embodiments the sodium acetate may be provided by including acetic acid and sodium acetate trihydrate in the solution. In certain particular embodiments, the composition comprises a phosphate.

It will be appreciated that the compstatin analog and/or additional active agent(s) can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

It will be understood that the pharmaceutically acceptable carriers, compounds, and preparation methods mentioned herein are exemplary and non-limiting. See, e.g., Remington: The Science and Practice of Pharmacy. 21st Edition. Philadelphia, PA. Lippincott Williams & Wilkins, 2005, for additional discussion of pharmaceutically acceptable compounds and methods of preparing pharmaceutical compositions of various types.

In some aspects, described herein are various doses, dosing regimens, compositions, and methods useful for treating patients by IVT injection, e.g., in a manner that is acceptable to physicians and patients in terms of the time and pressure required to deliver a given volume of the composition through a needle of a given inner diameter or gauge number. Gauge number describes the outer diameter of a hollow needle, with a higher gauge number indicating a smaller outer diameter. Generally, needles having a higher gauge number may be preferred by patients and physicians as they may be associated with (or may be perceived to be associated with) less pain and/or tissue damage as compared with needles having a lower gauge number. Inner diameter depends on both outer diameter and wall thickness. For standard needles, the higher the gauge number, the smaller the inner diameter (e.g., a 27 gauge needle has a larger inner diameter than a 29 gauge needle, which in turn has a larger inner diameter than a 30 gauge needle).

In some aspects, the present disclosure teaches particular utility of certain thin-walled needles for administration of a compstatin analog in accordance with the present invention. For example, in some embodiments a thin wall needle is used for intraocular (e.g., intravitreal) injection of a compstatin analog. Thin wall needles have identical outer diameters to standard needles but larger inner diameters for a given gauge. For example, a thin wall needle may have an internal diameter size that is the same as that of a standard needle of a gauge one to two numbers lower (e.g., a 29 gauge thin wall needle may have an internal diameter that is the same as that of a 27 gauge or 28 gauge standard needle but an outer diameter that is the same as that of a standard 29 gauge needle). An increase in internal diameter can result in a considerable increase in fluid flow for a given pressure and/or a considerable reduction in pressure needed to maintain a given flow. Lower pressure means that less injection force is needed to administer a composition of a given viscosity. In general, low injection force facilitates administration and is therefore typically a desirable feature. In some embodiments a thin wall needle has a given internal diameter that is uniform along the length of the needle. In some embodiments a thin wall needle has an internal diameter that varies along the length of the needle. For example, the diameter may be the same as that of a standard 29 gauge needle at one end of the needle and progress to the diameter of a standard 27 gauge needle at the other end. In some embodiments a microtapered needle may be used. In some embodiments a needle with a scalpel-like tip may be used. The length of the needle may vary. In some embodiments a short needle such as a 5 mm or 6 mm needle may be used. In some embodiments a needle having a length between 6 mm and 8 mm, or between 8 mm and 12 mm may be used. Suitable needles and syringes are available commercially, e.g., from Becton Dickinson and Company (BD), Terumo Corp., etc.

In some embodiments a composition having a given viscosity and/or concentration may be administered using a thin wall needle having a gauge that is one or two numbers higher than the gauge size that is preferably used when a standard needle is used to administer a composition of the same viscosity and/or concentration at a selected flow rate and/or with a selected injection force. For example, in some embodiments a composition that is preferably administered using a 25 gauge standard needle in order to attain a desired flow rate and/or injection force can be administered with such a flow rate and/or injection force using a 26 or 27 gauge thin wall needle. In some embodiments a composition that is preferably administered using a 27 gauge standard needle in order to attain a desired flow rate and/or injection force can be administered with such a flow rate and/or injection force using a 28 or 29 gauge thin wall needle. In some embodiments a composition that is preferably administered using a 29 gauge standard needle in order to attain a desired flow rate and/or injection force can be administered with such a flow rate and/or injection force using a 30 or 31 gauge thin wall needle.

In some embodiments a composition is administered using a syringe with one or more design features that reduce friction and/or required injection force, such as a relatively short barrel and/or relatively large plunger size.

In some embodiments, a composition, e.g., pegcetcoplan, is administered using a sterile 1-ml Luer-lock syringe. In some embodiments, a sterile 1-ml Luer-lock syringe has a 0.1 ml dose mark. In some embodiments, such a syringe is provided in a kit comprising a transfer needle (e.g., a filtered transfer needle) and/or needle for injection as described herein.

In some aspects, described herein are various doses, dosing regimens, compositions and methods of use for treating a patient in need of treatment for a complement-mediated eye disorder that affects the posterior segment of the eye, e.g., the retina, by intravitreal (IVT) administration of a compstatin analog. In some embodiments, the eye disorder is AMD, e.g., advanced AMD (geographic atrophy (GA) or neovascular AMD). In some embodiments a composition, e.g., a pharmaceutical composition, comprises a compstatin analog comprising a PEG having a molecular weight of about 40 kD (e.g., pegcetacoplan) at a concentration of about 150 mg/ml.

In some embodiments a dose for intravitreal injection of a compstatin analog (e.g., comprising two compstatin analog moieties and a PEG having a molecular weight of about 40 kD) is 5 mg-20 mg. In some embodiments the dose is about 10 mg. In some embodiments the dose is about 10 mg, about 11 mg, about 12 mg, about 13 mg, about 14 mg, about 15 mg, about 16 mg, about 17 mg, about 18 mg, about 19 mg, or about 20 mg. In certain particular embodiments the dose is about 15 mg. In some embodiments, any of the afore-mentioned doses is administered by intravitreal injection in a volume of between about 90 and about 110 microliters, e.g., in a volume of about 100 microliters.

In certain embodiments a composition, e.g., a composition that may be used for IVT administration, e.g., for treatment of AMD, comprises about 150 mg/ml compstatin analog (e.g., comprising a PEG of about 40 kD, e.g., pegcetacoplan), and one or more excipients selected from the group consisting of NaCl, trehalose, and sorbitol. In certain embodiments any of the compositions comprising about 150 mg/ml compstatin analog has a concentration between 140 mg/ml and 150 mg/ml, e.g., between 145 mg/ml and 155 mg/ml, e.g., between 148 mg/ml and 152 mg/ml, e.g., 150 mg/ml compstatin analog. In certain embodiments any of the compositions comprises about 20 mM sodium acetate. In certain embodiments any of the compositions has a pH between 4.8 and 5.2, e.g., about 5.0.

In some embodiments the composition comprises about 150 mg/ml compstatin analog and between 0.45% and 0.60% w/v NaCl, e.g., between 0.45% and 0.50%, between 0.50% and 0.55%, or between 0.55% and 0.60% NaCl. In certain embodiments the composition comprises between 0.51% NaCl and 0.54% NaCl. In certain embodiments the composition comprises between 0.52% NaCl and 0.53% NaCl. In particular embodiments the composition comprises 0.500% NaCl. In particular embodiments the composition comprises 0.505% NaCl. 0.510% NaCl. In particular embodiments the composition comprises 0.515% NaCl. In particular embodiments the composition comprises 0.520% NaCl. In particular embodiments the composition comprises 0.525% NaCl. In particular embodiments the composition comprises s 0.530% NaCl. In particular embodiments the composition comprises 0.535% NaCl. In particular embodiments the composition contains 0.540% NaCl. In particular embodiments the composition comprises 0.545% NaCl. In particular embodiments the composition comprises 0.550% NaCl.

In certain embodiments the composition comprises about 150 mg/ml compstatin analog and between 2.5% and 4.5% w/v sorbitol w/v, e.g., between 2.5% and 3.0%, between 3.0% and 3.5%, between 3.5% and 4.0%, or between 4.0% and 4.5% sorbitol. In particular embodiments the composition comprises 3.0% sorbitol. In particular embodiments the composition comprises 3.1% sorbitol. In particular embodiments the composition comprises 3.2% sorbitol. In particular embodiments the composition comprises 3.3% sorbitol. In particular embodiments the composition comprises 3.4% sorbitol. In particular embodiments the composition comprises 3.5% sorbitol.

In certain embodiments the composition comprises about 150 mg/ml compstatin analog and between 6.0% and 8.0% w/v trehalose, e.g., between 6.0% and 6.5%, between 6.5% and 7.0%, between 7.0% and 7.5%, or between 7.5% and 8.0% trehalose. In particular embodiments the composition comprises 6.5% trehalose. In particular embodiments the composition comprises 6.6% trehalose. In particular embodiments the composition comprises 6.7% trehalose. In particular embodiments the composition comprises 6.8% trehalose. In particular embodiments the composition comprises 6.9% trehalose. In particular embodiments the composition comprises 7.0% trehalose. In some embodiments, the composition comprises about 5.0% to about 6.0% trehalose, about 5.0% to about 5.5% trehalose, about 5.5% to about 6.0% trehalose, about 5.0% to about 5.3% trehalose, or about 5.2% to about 5.5% trehalose. In particular embodiments, the composition comprises about 5.3% to about 5.4% (e.g., about 5.38%) trehalose. In certain embodiments, the trehalose may be provided by including trehalose dehydrate in the solution.

In some embodiments, the osmolality of a solution comprising a compstatin analog may be measured at a dilution such that the concentration of compstatin analog is about 75 mg/ml or less. A solution comprising a compstatin analog and, optionally, an osmolality modifier, may be diluted with water by an appropriate dilution factor such that the concentration of the compstatin analog is 75 mg/ml or less, e.g., between 25 mg/ml and 40 mg/ml or between 40 mg/ml and 75 mg/ml. The osmolality of the resulting solution is measured and then multiplied by the dilution factor to correct for the dilution. For example, a solution having a compstatin analog concentration of 100 mg/ml may be diluted by a factor of 2, i.e., to a concentration of 50 mg/ml. The osmolality of the resulting solution is measured and multiplied by 2 to yield the osmolality of the original 100 mg/ml solution. As another example, a solution having a compstatin analog concentration of 150 mg/ml may be diluted by a factor of 2, i.e., to a concentration of 75 mg/ml. The osmolality of the resulting solution is measured and multiplied by 2 to yield the osmolality of the original 100 mg/ml solution.

In some embodiments a composition comprising a compstatin analog may have a selected osmolality. In some embodiments a composition comprising a compstatin analog, e.g., a composition for ocular, e.g., intravitreal, administration, has an osmolality of between 150 milliosmoles/kilogram (mOsm/kg) and 600 mOsm/kg, e.g., between 180 mOsm/kg and 500 mOsm/kg, e.g., between 200 mOsm/kg and 400 mOsm/kg. In certain embodiments a composition comprising a compstatin analog has an osmolality between 250 mOsm/kg and 380 mOsm/kg, e.g., between 275 mOsm/kg and 350 mOsm/kg. In certain embodiments a composition comprising a compstatin analog has an osmolality of between 275 mOsm/kg and 285 mOsm/kg, between 285 mOsm/kg and 295 mOsm/kg, between 295 mOsm/kg and 305 mOsm/kg, or between 305 mOsm/kg and 315 mOsm/kg. In particular embodiments the osmolality of the composition is 300 mOsm/kg. In some embodiments the osmolality of a composition may be measured using a vapor pressure depression osmometer. In some embodiments the osmolality of a composition may be measured using a membrane osmometer. In some embodiments the osmolality of a composition may be measured using a freezing point depression osmometer.

In some embodiments a dose of a composition comprising a compstatin analog is administered by ocular administration (e.g., IVT injection) once a month (e.g., 30 days), every 6 weeks, or every 2 months (i.e., every other month, (e.g., 60 days)). In some embodiments a dose of a composition comprising a compstatin analog is administered by IVT injection every 3 months, every 4 months, every 5 months, or every 6 months, or less frequently, e.g., every 9 months, every year). Thus in some embodiments a patient may receive between 1 and 6 injections per year, typically at approximately equal intervals. In some embodiments, a patient may receive between 6 and 12 injections per year, typically at approximately equal intervals. In some embodiments a patient is initially treated with monthly injections (e.g., for the first 3-6 months or the first 6-12 months), followed by less frequent administration (e.g., every 2, 3, 4, 5, or 6 months, or less frequently, e.g., every 9 months, every year).

In some embodiments, a pharmaceutical composition described herein includes pegcetacoplan at 15 mg/0.1 mL (150 mg/mL), as a sterile, aqueous, acetate-buffered trehalose solution for intravitreal administration. In some embodiments, such pharmaceutical composition is included in a single-use 2R vial containing 60 mg of pegcetacoplan in 0.4 mL solution, 75 mg of pegcetacoplan in 0.5 mL solution, 150 mg of pegcetacoplan in 1.0 mL solution, 45 mg of pegcetacoplan in 0.3 mL solution, or 30 mg of pegcetacoplan in 0.2 mL solution. In some embodiments, each vial is designed to deliver a single dose of 0.1 mL solution containing 15 mg of pegcetacoplan. Those skilled in the art, reading the present disclosure, will appreciate that, in accordance with standard practice in the field, a container containing a particular volume, as described herein my include an additional volume sufficient to permit the designated particular volume (e.g., unit dose) to be withdrawn from the container for administration.

In some embodiments, a pharmaceutical composition includes 15 mg/0.1 mL pegcetacoplan, 16-22 mM acetate buffer (e.g., about 16 mM, about 17 mM, about 18 mM, about 19 mM, about 20 mM, about 21 mM, or about 22 mM acetate buffer), 5% to 6% trehalose (e.g., about 5%, about 5.1%, about 5.2%, about 5.3%, about 5.4%, about 5.5%, about 5.6%, about 5.7%, about 5.8%, about 5.9%, or about 6.0% trehalose, e.g., about 5.38% trehalose), with a pH of about 4.8 to about 5.2 (e.g., about 5.0).

In some embodiments, a pharmaceutical composition includes the following, in a 0.1 mL volume: 15 mg Pegcetacoplan, about 5.5 mg to about 6.5 mg trehalose dihydrate (e.g., about 5.5 mg, about 5.6 mg, about 5.7 mg, about 5.8 mg, about 5.9 mg, about 6 mg, about 6.1 mg, about 6.2 mg, about 6.3 mg, about 6.4 mg, or about 6.5 mg trehalose dihydrate (e.g., about 5.95 mg trehalose dihydrate)), about 0.085 mg to about 0.095 mg glacial acetic acid (e.g., about 0.085 mg, about 0.0855 mg, about 0.086 mg, about 0.0865 mg, about 0.087 mg, about 0.0875 mg, about 0.088 mg, about 0.0885 mg, about 0.089 mg, about 0.0895 mg, about 0.09 mg, about 0.0905 mg, about 0.091 mg, about 0.0915 mg, about 0.092 mg, about 0.0925 mg, about 0.093 mg, about 0.0935 mg, about 0.094 mg, about 0.0945 mg, or about 0.095 mg glacial acetic acid (e.g., about 0.0895 mg glacial acetic acid)), about 0.03 mg to about 0.04 mg sodium acetate trihydrate (e.g., about 0.031 mg, about 0.032 mg, about 0.033 mg, about 0.034 mg, about 0.035 mg, about 0.036 mg, about 0.037 mg, about 0.038 mg, about 0.039 mg, or about 0.04 mg sodium acetate trihydrate (e.g., about 0.0353 mg sodium acetate trihydrate)), water for injection, and optionally sodium hydroxide and/or additional glacial acetic acid in an amount sufficient to adjust the pH of the pharmaceutical composition to about 4.8 to about 5.2 (e.g., about 5.0).

### VII. Treatment Methods

In some embodiments, a compstatin analog described herein, e.g., pegcetacoplan, is used to treat geographic atrophy (GA). In some embodiments, a compstatin analog described herein, e.g., pegcetacoplan, is administered to an eye of a subject having or suffering from GA. In some embodiments, one eye of the subject has GA, and pegcetacoplan is administered to that eye. In some embodiments, both eyes of the subject have GA, and pegcetacoplan is administered to both eyes. In some embodiments, one eye of the subject has GA and one eye has wet AMD, and pegcetacoplan is administered to the eye having GA. In some embodiments, one eye of the subject has GA, and a second eye of the subject has wet AMD but not GA, and pegcetacoplan is administered to the first eye. In some embodiments, one eye of the subject has GA, and a second eye of the subject has wet AMD and has GA, and pegcetacoplan is administered to the first eye. In some embodiments, one eye of the subject has GA, and a second eye of the subject has wet AMD and has GA, and pegcetacoplan is administered to the second eye. In some embodiments, one eye of the subject has GA, and a second eye of the subject has wet AMD and has GA, and pegcetacoplan is administered to the first eye and to the second eye.

In some embodiments, administration of a compstatin analog described herein, e.g., pegcetacoplan, to a subject maintains, reduces loss of, and/or improves visual function in the subject. In some embodiments, visual function is assessed by one or more of microperimetry, monocular maximum reading speed, Functional Reading Independence (FRI) Index, best-corrected visual acuity (BCVA) (e.g., normal luminance BCVA (NL-BCVA) or low luminance BCVA (LL-BCVA)). In some embodiments, administration of a compstatin analog described herein, e.g., pegcetacoplan, to a subject maintains, reduces loss of, and/or improves visual function in the subject by at least 10%, 20%, 30%, 40%, 50%, or more, relative to a control (e.g., as assessed by one or more of microperimetry, monocular maximum reading speed, Functional Reading Independence (FRI) Index, best-corrected visual acuity (BCVA) (e.g., normal luminance BCVA (NL-BCVA) or low luminance BCVA (LL-BCVA)). For example, in some embodiments, visual function of the eye is assessed by microperimetry, and after administration of a compstatin analog described herein, e.g., pegcetacoplan, mean threshold sensitivity of the eye is about 0.25, about 0.5, about 0.75, or about 1 db higher, relative to a control. In some embodiments, visual function of the eye is assessed by microperimetry, and after administration of a compstatin analog described herein, e.g., pegcetacoplan, the subject exhibits fewer number of scotomatous points, relative to a control. In some embodiments, the eye of the subject has a geographic atrophy lesion, and microperimetry is assessed within an area peripheral to a lesion border, e.g., within an area within about 500 microns (e.g., within about 400 microns, within about 300 microns, within about 250 microns, within about 200 microns, within about 150 microns) of each side of the lesion border. In some embodiments, a control is level of visual function in a sham-treated subject (e.g., an untreated subject having the same level of risk and/or stage or severity of GA as the treated subject) or an average level of visual function in an untreated subject population (e.g., an untreated subject population having the same level of risk and/or stage or severity of GA as the treated subject).

In some embodiments, a compstatin analog described herein, e.g., pegcetacoplan, is administered, e.g., intravitreally, to the eye of the subject as a pharmaceutical composition that includes pegcetacoplan at 15 mg/0.1 mL (150 mg/mL). In some embodiments, the composition is administered to the eye of the subject once a month (e.g., 30 days), e.g., for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer. In some embodiments, the composition is administered to the eye of the subject every other month (e.g., 60 days), e.g., for about 8 weeks, about 16 weeks, about 24 weeks, about 32 weeks, about 40 weeks, about 48 weeks, about 1.2 years, 1.4 years, 1.6 years, 1.8 years, 2 years, 3 years, 4 years, 5 years, or longer. In some embodiments, a specific improvement (e.g., a statistically significant or clinically significant improvement) of one or more GA symptoms or parameters is achieved in the subject, e.g., one or more target levels described herein is achieved.

In some embodiments, a subject may be monitored and/or evaluated for elevated intraocular pressure (IOP) prior to administration, e.g., intravitreal injection, of a composition described herein. In some embodiments, IOP is measured using tonometry. In some embodiments, a subject may be administered ocular hypotensive medication to lower IOP. In some embodiments, prior to administration, e.g., intravitreal injection of a composition described herein, IOP is about ≤21 mmHg. In some embodiments, an eye of the subject may be softened and/or decompressed prior to administration, e.g. intravitreal injection, of a composition described herein. In some embodiments, an eye of the subject may be softened and/or decompressed using a cotton-tipped applicator. In some embodiments, a cotton-tipped applicator may be saturated with sterile topical anesthetic drops. In some embodiments, an eye of the subject may be softened and/or decompressed by pushing against the globe with the cotton-tipped applicator at the planned injection site for between about 30 to about 60 seconds. In some embodiments, softening and/or decompressing an eye of a subject prior to administration, e.g., intravitreal injection, of a composition described herein may prevent and/or minimize an increase in IOP.

In some embodiments, methods of the disclosure include diagnosing the subject as suffering from or at risk of geographic atrophy (GA), e.g., GA secondary to age related macular degeneration (AMD), and/or selecting the subject for treatment with pegcetacoplan. In some embodiments, diagnosing may comprise performing fundus autofluorescence (FAF) and/or optical coherence tomography (OCT). In some embodiments, the subject is determined to have foveal GA (e.g., in one or both eyes) and is selected for treatment. In some embodiments, the subject is determined to have extrafoveal GA (e.g., solely extrafoveal GA) (e.g., in one or both eyes) and is selected for treatment. In some embodiments, the subject is selected for treatment if total GA area is ≥2.5 mm² (e.g., ≥2.5 mm² and ≤17.5 mm²).

In some embodiments a method of the disclosure may comprise assessing foveal light sensitivity of one or both eyes of a subject. In some embodiments such assessment may comprise performing microperimetry. In some embodiments the subject retains foveal light sensitivity in at least one eye at the time treatment with pegcetacoplan is initiated. In some embodiments the subject retains foveal light sensitivity in an eye at the time treatment of that eye with pegcetacoplan is initiated. In some embodiments the subject has reduced foveal light sensitivity in an eye relative to a control at the time treatment of that eye with pegcetacoplan is initiated.

In some embodiments an eye treated with pegcetacoplan by monthly IVT injection has a risk of about 6% or less per year for developing exudation. In some embodiments an eye treated with pegcetacoplan by every other month IVT injection has a risk of less than about 4% per year for developing exudation. In some embodiments the risk of developing exudation for an eye treated with pegcetacoplan by monthly IVT injection is about 2.5 (or less) times that of a control eye not treated with pegcetacoplan, e.g., between about 2.0 and about 2.5 times that of a control eye not treated with pegcetacoplan, e.g., over a specified time period such as a year. In some embodiments the risk of developing exudation for an eye treated with pegcetacoplan by monthly IVT injection is about 1.7 (or less) times that of a control eye not treated with pegcetacoplan, e.g., between about 1.5 and about 1.7 times that of a control eye not treated with pegcetacoplan, e.g., over a specified time period such as a year.

Methods described herein can include preparing and/or providing a report, such as in electronic, web-based, or paper form. The report can include one or more outputs from a method described herein, e.g., a subject's response to a treatment described herein. In some embodiments, a report is generated, such as in paper or electronic form, which identifies one or more endpoints described herein for a subject, and optionally, a recommended course of therapy. In some embodiments, the report includes an identifier for the subject. In some embodiments, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a subject, e.g., identified in the report. For example, the report can include information, or a recommendation, on the administration of a compstatin analog described herein, e.g., pegcetacoplan, to the subject. The report can be delivered, e.g., to an entity described herein, within 7, 14, 21, 30, or 45 days from performing a method described herein.

In some embodiments, a report is generated to memorialize each time a subject is assessed using a method described herein. The subject can be reevaluated at intervals, such as every month, every two months, every six months or every year, or more or less frequently, to monitor the subject for responsiveness to compstatin analog, e.g., pegcetacoplan, and/or for an improvement in one or more GA symptoms, e.g., described herein. In some embodiments, the report can record at least the treatment history of the subject.

In some embodiments, the method further includes providing a report to another party. The other party can be, for example, the subject, a caregiver, a physician, an oncologist, a hospital, clinic, third-party payor, insurance company or a government office.

The disclosure encompasses administration of a compstatin analog in combination with additional therapy. Such additional therapy may include administration of any agent(s) used in the art or potentially useful for treating a subject suffering from the disease. For example, in some embodiments a compstatin analog is administered in combination with a C5 inhibitor (e.g., eculizumab or any of the other C5 inhibitors mentioned herein or known in the art) to a patient, e.g., a patient with AMD. In some embodiments a compstatin analog is administered in combination with an anti-vascular endothelial growth factor (VEGF) agent to a subject, e.g., a subject with wet AMD. Anti-VEGF agents include antibodies that bind to VEGF such as ranibizumab (Lucentis), bevacizumab (Avastin), brolucizumab (Novartis), faricimab (Roche), and polypeptides comprising a soluble portion of VEGF receptor such as aflibercept (Eylea, also known as VEGF-Trap) and conbercept (Chengdu Kanghong Biotech). In particular embodiments the anti-VEGF agent is ranibizumab or aflibercept.

In some embodiments a subject treated with a compstatin analog, e.g., pegcetacoplan, may develop exudation in the treated eye. In some embodiments a subject who exhibits evidence of exudation in an eye being treated with pegcetacoplan may be started on therapy with an anti-VEGF agent administered to said eye. In some embodiments a method of treatment comprises administering pegcetacoplan to an eye suffering from GA and evaluating said eye for development of exudation (e.g., related to active CNV). Evaluating may comprise performing fundus examination and/or optical coherence tomography (OCT). In some embodiments, a subject may be determined to have exudation based on detection of, e.g., subretinal fluid, intraretinal fluid, cystoid macular edema, and/or serous pigment epithelial detachment. In some embodiments evaluating for presence of exudation may comprise performing fluorescein angiography (FA) and/or optical coherence tomography angiography (OCT-A). In some embodiments a subject may be confirmed to have exudation by FA and/or OCT-A. In some embodiments an evaluation procedure may be performed when the subject is visiting a health care provider, e.g., an eye care professional, for administration of pegcetacoplan. In some embodiments, a subject may be instructed to report to a health care provider, e.g., an eye care professional, any symptoms of exudation. In some embodiments a subject determined to exhibit exudation (e.g., onset of exudation) in an eye treated with pegcetacoplan may be started on treatment of said eye with an anti-VEGF agent. In some embodiments a method of treatment comprises determining that an eye being treated with pegcetacoplan exhibits evidence of exudation and administering an anti-VEGF agent to said eye. In some embodiments a method of treatment comprises determining that an eye having wet AMD exhibits GA and administering pegcetacoplan and an anti-VEGF agent to said eye. In some embodiments a method of treatment comprises determining that an eye has wet AMD and GA and administering pegcetacoplan and an anti-VEGF agent to said eye. In some embodiments an anti-VEGF agent may be an agent that is approved for treatment of neovascular AMD and is administered according to its approved dosing regimen for treatment of neovascular AMD as described in the prescribing information for such anti-VEGF agent. In some embodiments an eye may be treated by IVT injection with pegcetacoplan and an anti-VEGF agent on the same day. In some embodiments an eye may be treated by IVT injection with pegcetacoplan and an anti-VEGF agent on different days. In embodiments wherein an eye is treated with pegcetacoplan and an anti-VEGF agent on the same day, the anti-VEGF agent may be administered before administration of pegcetacoplan. In some embodiments administration of the anti-VEGF agent and pegcetacoplan are administered at least 30 minutes apart on the same day. In some embodiments, administration of the anti-VEGF agent is administered at least 30 minutes before administration of pegcetacoplan. In some embodiments, wherein an eye is treated with pegcetacoplan and an anti-VEGF agent on the same day, the anti-VEGF agent may be administered after administration of pegcetacoplan. In some embodiments, administration of the anti-VEGF agent and pegcetacoplan are administered at least 30 minutes apart on the same day. In some embodiments, administration of the anti-VEGF agent is administered at least 30 minutes after administration of pegcetacoplan.

When two or more therapies (e.g., compounds or compositions) are used or administered “in combination” with each other, they may be given at the same time, within overlapping time periods, or sequentially (e.g., separated by up to 2 weeks in time, or more, e.g., separated by up to about 4, 6, 8, or 12 weeks in time), in various embodiments of the invention. They may be administered via the same route or different routes. In some embodiments, the compounds or compositions are administered within 48 hours of each other. In some embodiments, a compstatin analog can be given prior to or after administration of the additional compound(s), e.g., sufficiently close in time that the compstatin analog and additional compound(s) are present at useful levels within the body at least once. In some embodiments, the compounds or compositions are administered sufficiently close together in time such that no more than 90% of the earlier administered composition has been metabolized to inactive metabolites or eliminated, e.g., excreted, from the body, at the time the second compound or composition is administered.

In some embodiments, a composition that includes both a compstatin analog and additional compound(s) is administered.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. The example is provided for illustrative purposes only. It is not to be construed as limiting the scope or content of the disclosure in any way.

### EXEMPLIFICATION

### Example 1—Phase III, Multi-Center, Randomized, Double-Masked, Sham-Controlled Studies to Compare the Efficacy and Safety of Intravitreal APL-2 Therapy with Sham Injections in Patients with Geographic Atrophy (GA) Secondary to Age-Related Macular Degeneration (AMD)

This Example describes results from two Phase 3 multi-center, randomized, double-masked, sham-controlled studies (“DERBY” and “OAKS”) to compare the efficacy and safety of intravitreal APL-2 (pegcetacoplan, also referred to as “Study Drug” in this Example) therapy with sham injections in patients with geographic atrophy (GA) secondary to age-related macular degeneration (AMD).

### Methods

### Patient Selection

To participate in the studies, subjects must have been diagnosed with GA of the macula secondary to AMD in the study eye. At screening, male and female subjects were required to fulfill all of the following inclusion criteria to be eligible for participation in the study. If both eyes met the inclusion criteria, the eye with the worst normal luminance visual acuity at the screening visit was designated as the study eye. If both eyes had the same visual acuity, the right eye was selected as the study eye. Ocular-specific inclusion criteria applied to the study eye only, unless otherwise specified.

### Inclusion Criteria

1. Age≥60 years.
2. Normal Luminance best corrected visual acuity (BCVA) of 24 letters or better using Early Treatment Diabetic Retinopathy Study (ETDRS) charts (approximately 20/320 Snellen equivalent).
3. Clinical diagnosis of GA of the macula secondary to AMD as determined by the investigator and confirmed by the reading center.
4. The GA lesion must have met the following criteria as determined by the central reading center's assessment of FAF imaging at screening:
   a. Total GA area must be ≥2.5 and ≤17.5 mm² (1 and 7 disk areas [DA] respectively).
   b. If GA was multifocal, at least 1 focal lesion must be ≥1.25 mm² (0.5 DA), with the overall aggregate area of GA as specified above in 4a.
   c. The entire GA lesion must be completely visualized on the macula centered image and must be able to be imaged in its entirety and not contiguous with any areas of peripapillary atrophy.
   d. Presence of any pattern of hyperautofluorescence in the junctional zone of GA. Absence of hyperautofluorescence (ie, pattern=none) was exclusionary.
5. Adequate clarity of ocular media, adequate pupillary dilation, and fixation to permitted the collection of good quality images as determined by the investigator.
6. Met the following criteria related to microperimetry:
   a. Able to detect fixation target.
   b. Total elapsed time to complete the 10-2 68-point exam was ≤30 minutes in duration.
   c. Fixation losses must be≤20%.
   d. Subject was willing and able to undertake microperimetry assessment in the opinion of the investigator.
7. Female subjects must have been:
   a. Women of non-childbearing potential (WONCBP), or
   b. Women of childbearing potential (WOCBP) with a negative serum pregnancy test at screening and agreed to use protocol defined methods of contraception for the duration of the study and refrain from breastfeeding for the duration of the study.
8. Males with female partners of childbearing potential must agree to use protocol defined methods of contraception and agree to refrain from donating sperm for the duration of the study.
9. Willing and able to give informed consent and to comply with the study procedures and assessments.

Subjects were excluded from the studies based on the following exclusion criteria. Ocular specific exclusion criteria applied to the study eye only, unless otherwise specified.

### Exclusion Criteria

1. GA secondary to a condition other than AMD such as Stargardt disease, cone rod dystrophy, or toxic maculopathies like plaquenil maculopathy in either eye.
2. Spherical equivalent of the refractive error demonstrating >6 diopters of myopia or an axial length >26 mm.
3. Any history or active CNV in the study eye, associated with AMD or any other cause, including any evidence of retinal pigment epithelium rips or evidence of neovascularization anywhere based on SD-OCT imaging and/or fluorescein angiography as assessed by the reading center.
4. Presence of an active ocular disease that in the opinion of the investigator compromised or confounded visual function, including but not limited to, uveitis, other macular diseases (e.g., clinically significant epiretinal membrane [ERM], full thickness macular hole) or uncontrolled glaucoma/ocular hypertension). Benign conditions in the opinion of the investigator such as peripheral retina dystrophy were not exclusionary.
5. Intraocular surgery (including lens replacement surgery) within 3 months prior to randomization.
6. History of laser therapy in the macular region.
7. Aphakia or absence of the posterior capsule. Note: YAG laser posterior capsulotomy for posterior capsule opacification done at least 60 days prior to screening was not exclusionary.
8. Any ocular condition other than GA secondary to AMD that may have required surgery or medical intervention during the study period or, in the opinion of the investigator, could have compromised visual function during the study period.
9. Any contraindication to IVT injection including current ocular or periocular infection.
10. History of prior intravitreal injection.
11. Unable to perform microperimetry reliably in the opinion of the investigator.
12. Prior participation in another interventional clinical study for intravitreal therapies in either eye (including subjects receiving sham).
13. Prior participation in another interventional clinical study for geographic atrophy in either eye including investigational oral medication and placebo.
14. Participation in any systemic experimental treatment or any other systemic investigational new drug including within 6 weeks or 5 half-lives of the active ingredient (whichever was longer) prior to the start of study treatment. Note: clinical trials solely involving observation, over-the-counter vitamins, supplements, or diets were not exclusionary.
15. Medical or psychiatric conditions that, in the opinion of the investigator, would make consistent follow-up over the 24-month treatment period unlikely, or would make the subject an unsafe study candidate.
16. Any screening laboratory value (hematology, serum chemistry or urinalysis) that in the opinion of the investigator was clinically significant and not suitable for study participation. 17. Known hypersensitivity to fluorescein sodium for injection or hypersensitivity to pegcetacoplan or any of the excipients in pegcetacoplan solution.

### Study Design

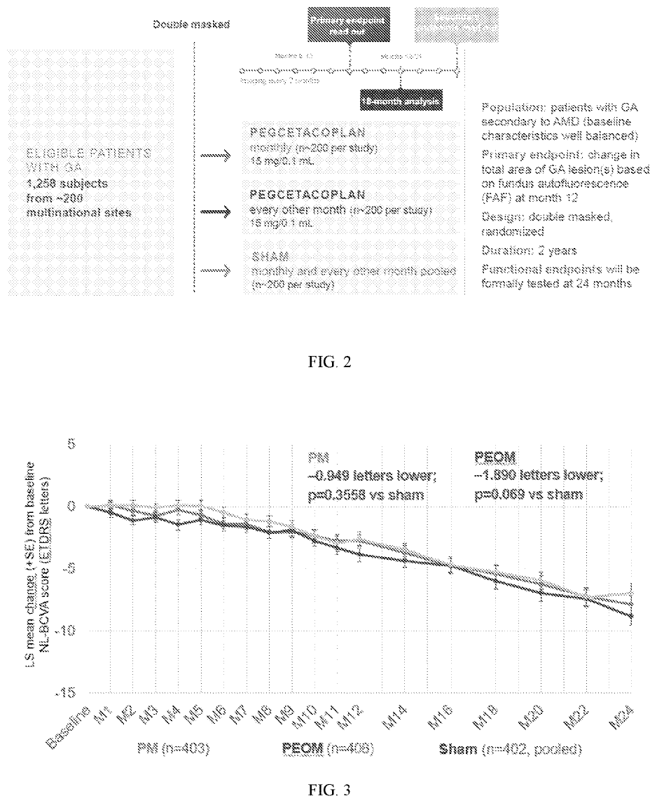

The design of the DERBY and OAKS studies are schematically depicted in FIG. 2. Each subject was assigned a unique screening number after signing the informed consent. Subjects who completed the study screening assessments and met all the eligibility criteria entered the study and were randomized on Day 1. As part of the screening process, the reading center evaluated FAF, OCT, Digital Color Fundus Photograph (DCFP), Near Infrared Reflectance (NIR), and fluorescein angiography (FA) to provide an objective assessment of subject eligibility. Subjects were randomized 2:2:1:1 to receive treatment with Study Drug monthly (PM), Study Drug every other month (PEOM), sham injection monthly (SM) or sham injection every other month (SEOM), respectively.

All randomized subjects returned every month to the clinical site for assessments and additional pegcetacoplan or sham injections according to their randomization scheme until Month 12. From Month 12 onwards, subjects return to the clinical site based on their randomized treatment schedule (monthly or EOM) and follow the treatment regimen and assessments outlined in the Visit Schedule until Month 24. Safety was assessed throughout the study by a number of evaluations including: monitoring of AEs, preinjection and postinjection monitoring, blood and urine samples collected, physical examination, vital signs, and follow-up phone calls. Blood samples were also be collected for anti-therapeutic antibodies, genotyping, and clinical repository (if the subject consented to this portion).

### Study Treatments

The Study Drug was pegcetacoplan (also referred to as “APL-2”) (see FIG. 1), which was provided for intravitreal injection at 15 mg/0.1 mL (150 mg/mL) as a sterile, isotonic solution of pegcetacoplan in acetate-buffer, pH 5.0, containing trehalose, supplied in stoppered glass vials.

### Screening Period—Within 28 Days Prior to Randomization/Treatment (Day −28 to −1)

### Visit 1—All Subjects

Note: All ophthalmic procedures (including imaging) were performed on both eyes, except where specified. Demographic information, significant medical/surgical history within the previous 5 years, invasive ocular procedures within the previous 5 years, and concomitant medications used within 30 days prior to screening were collected (including vitamins and all over-the-counter as well as prescription medications). Complete smoking/tobacco history was also collected.

Subject eligibility was then be determined by reviewing the inclusion/exclusion criteria and the study eye was selected. Prior to the administration of fluorescein, blood and urine was collected for safety labs (including blood for human chorionic gonadotropin/follicle-stimulating hormone/luteinizing hormone [HCG/FSH/LH], if applicable) and vital signs along with a physical examination including weight and height was performed.

A complete ophthalmic exam including slitlamp exam of the cornea, iris, anterior chamber, aqueous reaction (cells and flare), dilated fundus exam of the vitreous and retina, and intraocular pressure (IOP) measurement. Normal luminance BCVA was performed prior to dilating the eyes. Images were captured as outlined in the Visit Schedule (FAF, NIR, DCFP, FFA) and forwarded to the reading center for determination of eligibility if applicable. Mesopic microperimetry was performed on both eyes post dilation of the eyes and forwarded to the reading center.

Randomization/Initial Treatment—Day 1—within 28 Days of Screening

### Visit 2—All Groups

At this visit, all inclusion/exclusion criteria was reviewed prior to randomization and dosing, including the determination of eligibility by the reading center. Subjects were randomized using the Interactive Web Response (IWR) System. A complete ophthalmic exam including slitlamp exam of the cornea, iris, anterior chamber, lens, and aqueous reaction (cells and flare), dilated fundus exam of the vitreous and retina, and IOP measurement were performed and imaging collected as per the Visit Schedule.

All assessments were performed on the same day. All study visits were scheduled and projected based on the Day 1 visit date.

Blood was drawn for anti-pegcetacoplan antibodies.

Prior to dilating the eyes, all functional tests were performed (NL-BCVA, LL-BCVA, and MNREAD or Radner Reading Charts [in select sites]). Subjects at select sites were trained on how to use the home-based digital applications for visual function and reading speed if the subject decided to participate in this portion. This training occurred after completion of all functional tests (NL-BCVA, LL-BCVA). Tests completed using the digital application at select sites (optional) were completed prior to dilating the eyes but after completion of all functional tests and quality of life measures (NL-BCVA, LL-BCVA, MNREAD or Radner Reading Charts [in select sites], NEI VFQ-25, and FRI). The subject was instructed to take the electronic device home and to complete the digital application weekly on the same day each week, if possible. The quality of life measures (NEI VFQ-25 and FRI) was administered by the masked site staff.

Imaging performed included FAF, SD-OCT, OCT-A (select sites), endothelial cell count (select sites) and NIR and sent to the reading center for evaluation.

Study drug or sham injection was performed by the unmasked physician as described herein and the study eye was monitored post injection. Subjects received a single dose of 15 mg pegcetacoplan/0.1 mL or sham injection intravitreally. A follow-up phone call was scheduled with the subject 4±2 days after randomization Day 1 to assess for any AEs.

### Treatment Phase—24 Months

### Months 1-12

### Visits 3-14 (Monthly and Every-Other Month Group)

During this phase, there were clinic visits every month. Dosing and assessments occurred monthly in the monthly pegcetacoplan and sham injection treatment arms. Dosing occurred every other month in the EOM pegcetacoplan and sham injection treatment arms, however, the subjects returned monthly for assessments (with no dose given).

A complete ophthalmic exam including slitlamp exam of the cornea, iris, anterior chamber, lens, and aqueous reaction (cells and flare), dilated fundus exam of the vitreous and retina, and IOP measurement were performed and imaging collected as per the Visit Schedule.

Blood was drawn for safety labs, anti-pegcetacoplan antibodies, and genotyping (Month 2 only) prior to the administration of fluorescein as per the Visit Schedule. Samples were collected for the clinical repository for those subjects that consent to this portion.

Prior to dilating the eyes, all functional tests were performed (NL-BCVA, LL-BCVA, and MNREAD or Radner Reading Charts [in select sites]). Tests completed using the digital application at select sites (optional) were completed prior to dilating the eyes but after completion of all functional tests and quality of life measures (NL-BCVA, LL-BCVA, MNREAD or Radner Reading Charts [in select sites], NEI VFQ-25, and FRI). At select sites the subject was instructed to take the electronic device home and to complete the digital application weekly on the same day each week, if possible. The subject was instructed to bring back the electronic device for the visits specified in the schedule of events. The quality of life measures (NEI VFQ-25 and FRI) was administered by the masked site staff.

Mesopic microperimetry was performed on both eyes (where specified) post dilation of the eyes and forwarded to the reading center. Imaging was performed including FAF, FFA, SD-OCT, OCT-A (select sites), endothelial cell count (select sites) and NIR and sent to the reading center for evaluation per the visit schedule.

### Months 13-24

### Visits 15-26 (Monthly Group) and 15-20 (Every-Other-Month Group)

During this phase, clinic visits follow treatment designation (i.e., the monthly subjects return monthly for dosing and assessments and the EOM group returned EOM for dosing and assessments). At select sites, subjects complete the home-based digital assessments weekly during this period.

A complete ophthalmic exam including slitlamp exam of the cornea, iris, anterior chamber, lens, and aqueous reaction (cells and flare), dilated fundus exam of the vitreous and retina, and IOP measurement is performed and imaging is collected as per the Visit Schedule. Blood is drawn for safety labs, genotyping, and anti-pegcetacoplan antibodies prior to the administration of fluorescein as per the Visit Schedule. Samples are collected for the clinical repository for those subjects that consent to this portion.

Prior to dilating the eyes, all functional tests are performed (NL-BCVA, LL-BCVA, and MNREAD or Radner Reading Charts [in select sites]). The quality of life measures (NEI VFQ-25 and FRI) is administered by the masked site staff.

Mesopic microperimetry is performed on both eyes (where specified) post dilation of the eyes and forwarded to the reading center. Imaging is performed including FAF, FFA, SD-OCT, OCT-A (select sites), endothelial cell count (select sites) and NIR and sent to the reading center for evaluation per the visit schedule.

The completion of the 24-month study period occurs approximately 30 days (monthly treatment group) and approximately 60 days (EOM treatment group) after the last visit at which Study Drug is administered. The period following last dose of Study Drug is sufficient to evaluate the safety of pegcetacoplan. At the end of the 24-month study period, subjects have the option to enroll in a separate open-label study.

### Measures of Clinical Efficacy

The primary endpoint in the studies was the change from baseline to Month 12 in total area of GA lesion(s) in the study eye (in mm²) based on Fundus Autofluorescence (FAF). Key secondary endpoints included change from baseline in the mean threshold sensitivity of all points (study eye) assessed by mesopic microperimetry at Month 24; change from baseline in monocular maximum reading speed (study eye), at Month 24 as assessed by MNRead or Radner reading charts (in select sites); change from baseline in mean FRI Index score (subject-level assessment) at Month 24; and change from baseline in NL-BCVA score (study eye) at Month 24 as assessed by Early Treatment Diabetic Retinopathy Study (ETDRS) chart. Additional secondary endpoints included change from baseline in LL-BCVA score (study eye) over time as assessed by ETDRS chart; change from baseline in the total area of GA lesion(s) in the study eye (in mm²) as assessed by FAF over time other than Month 12; change from baseline in monocular critical print size (study eye), as assessed by MNRead or Radner reading charts (in select sites) over time; change from baseline in the NEI VFQ-25 distance activity subscale score (in select sites) (subject level assessment) over time; change from baseline in the number of scotomatous points assessed by mesopic microperimetry over time; and change from baseline in the mean threshold sensitivity within 500 microns outside the GA lesion, (perilesional points) assessed by mesopic microperimetry over time.

Safety endpoints included incidence and severity of ocular and systemic treatment-emergent adverse events; incidence of ADA directed against pegcetacoplan peptide or PEG; incidence of new active CNV in the study eye; incidence of new onset of subclinical CNV in the study eye; incidence of subjects who lost letters based on NL-BCVA categories (≥15, ≥15-<30, ≥30 ETDRS letters); change/shift from baseline in clinical labs and incidence of abnormal lab values; change from baseline in vital signs and incidence of abnormal vital sign results; shift from baseline in ocular examination assessments including slit-lamp examination and indirect ophthalmoscopy; change from baseline in Intra Ocular Pressure (IOP) and incidence of IOP above specified thresholds; and change/shift from baseline in ocular imaging assessments (including specular microscopy; select sites).

### Statistical Analysis

Two sham treatment arms (SM and SEOM) were pooled into a single control (Sham) group for analyses.

The Intent-to-treat (ITT) set consisted of all randomized subjects. Subjects were analyzed in the treatment arm assigned at randomization.

The modified ITT (mITT) set consisted of all randomized subjects who received at least one injection of pegcetacoplan or sham and had baseline and at least one post-baseline value of GA lesion area in the study eye as assessed by FAF. Subjects were analyzed in the treatment arm assigned at randomization.

The Safety set consisted of all subjects randomized who received at least one injection of pegcetacoplan or sham. Subjects were analyzed according to the actual treatment received. In the case a subject received an incorrect injection of study medication than what they were randomized to, subjects were presented under the corresponding pegcetacoplan arm if they received at least one injection of pegcetacoplan during the study and were only be presented under the corresponding sham arm if they did not receive any injections of pegcetacoplan. This population was used for all safety analyses.

The Per-Protocol (PP) sets will be identified separately for Month 12 and Month 24 analysis, respectively (i.e., Month 12 PP set and Month 24 PP set). The PP sets consisted of all mITT subjects who had a valid GA lesion area assessment for either Month 10 or 12 (Month 12 PP set) or have a valid GA lesion area assessment for at least one of Month 18, 20, 22, 24 (Month 24 PP set) and who follow the protocol without any major deviation(s) that could affect the primary efficacy data. A valid GA lesion area assessment is defined as a non-missing measured GA lesion area assessment at a given timepoint where at least 75% of the expected injections over the course of participation ahead of the given timepoint have been received by the subject. For example, a valid GA lesion measurement at Month 10 in the Monthly arm would be a result available at Month 10 with no more than 2 missed injections prior to the Month 10 assessment (10 scheduled before Month 10, 2 missed=80% compliance); a valid GA lesion measurement at Month 20 in the every other month (EOM) arm would be a result available at Month 20 with no more than 2 missing injections prior to the Month 20 assessment (10 scheduled before Month 20, 2 missed=80% compliance).

Efficacy analysis including primary, key secondary, secondary, and exploratory analysis will be performed primarily using the mITT set, with subjects grouped according to the treatment assigned at randomization. Available data from all randomized subjects regardless of adherence to the protocol will be included in the efficacy analyses; this includes data from subjects who discontinued study drug early but continued with study assessments. All efficacy data will be listed for the ITT set.

Unless otherwise noted, hypothesis testing and estimation of treatment effects will be performed with a mixed effect model for repeated measure (MMRM) that includes data from all three treatment arms (PM, PEOM, and Sham). The Sham arm will represent the pool of the two sham treatment groups: SM and SEOM (i.e., the two sham arms will be pooled into a single “control” group). All hypothesis tests for efficacy endpoints will be two-sided.

Unless otherwise noted, analysis of efficacy endpoints (primary, key secondary, secondary, and exploratory) in the overall population will be adjusted for the following randomization stratification factors (actual status) and baseline covariates:

For primary efficacy endpoint: presence of CNV in the fellow eye (yes; no); baseline GA lesion area (<7.5 mm² or ≥7.5 mm²).

For key secondary, and secondary endpoints: presence of CNV in the fellow eye (yes; no); baseline GA lesion area (<7.5 mm² or ≥7.5 mm²); baseline value of the endpoint.

Of note, the randomization stratification factors were assessed at the screening visit. In the event of a change in status in the GA lesion area between the screening visit and the baseline visit (i.e., GA lesion area<7.5 mm² at screening to ≥7.5 mm²) then the baseline status will be used.

### Results

### Efficacy

As shown in FIG. 3, no statistically significant differences were observed across study arms on key secondary endpoints at 24 months.

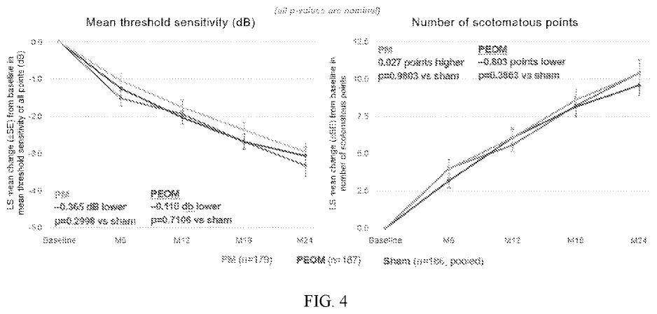

As shown in FIG. 4, no differences in microperimetry assessment were observed at 24 months across study arms. Microperimetry was assessed using iCare MAIA Microperimeter using 25 Hz eye-tracking with confocal SLO imaging.

A post hoc microperimetry perilesional analysis was performed on patients in the OAKS study to assess whether pegcetacoplan preservation of retinal tissue was reflected in preserved photoreceptor function in retina near GA lesion borders at baseline. Geographic atrophy lesion borders grow at approximately 100-150 microns per year. An assessment was performed of visual function of cells within 250 microns on either side of the lesion border, which are at the highest risk of permanently losing visual function as lesions expand. Reduction of retinal sensitivity as measured by microperimetry reflects loss of function. At 24 months, positive trends in microperimetry measures were demonstrated (all p-values nominal), as discussed below.

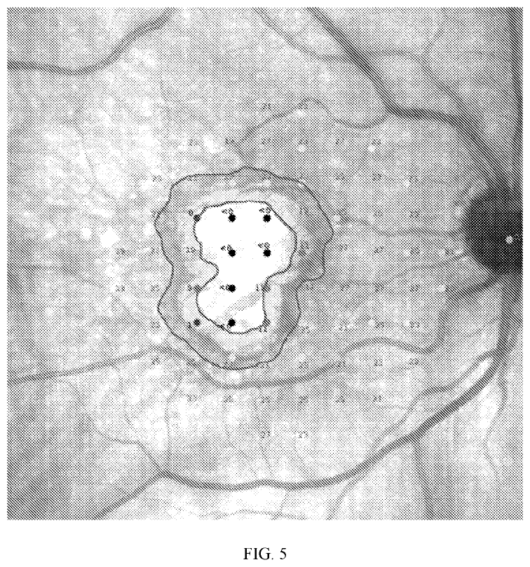

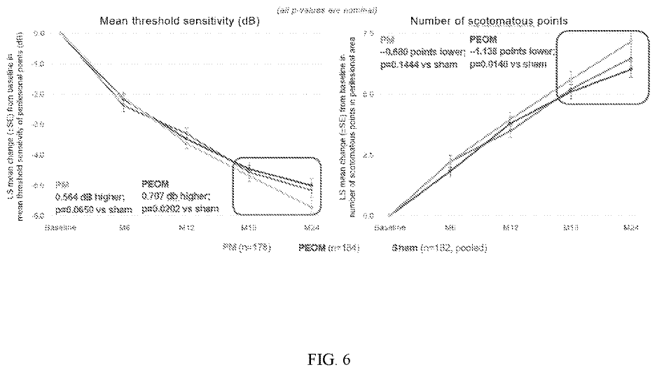

Perilesional area (area within 250 microns of each side of GA lesion border) was created on fundus autofluorescence for each patient, and microperimetry endpoints were assessed within this region (68 spots on grid). In this analysis, approximately 21-22 points (out of 68 total—about one-third) qualified as “peri-lesional” using the 500 micron zone definition (250 inside and 250 outside of GA lesion border). A representative image is depicted as FIG. 5. As shown in FIG. 6, the post hoc microperimetry perilesional analysis signaled functional preservation with pegcetacoplan treatment, consistent with less photoreceptor loss over time. Patients in both monthly (0.564 dB; p=0.0650) and every-other-month (EOM) (0.707 dB; p=0.0202) pegcetacoplan treatment groups lost less retinal sensitivity compared to the sham group over 24 months with increasing effects over time. Scotomatous points measure areas of the retina that have lost all light sensitivity and therefore are no longer functioning. Patients in both monthly (−0.680 points; p=0.1444) and EOM (−1.138 points; p=0.0140) pegcetacoplan treatment groups had fewer new scotomatous points compared to sham over 24 months with increasing effects over time.

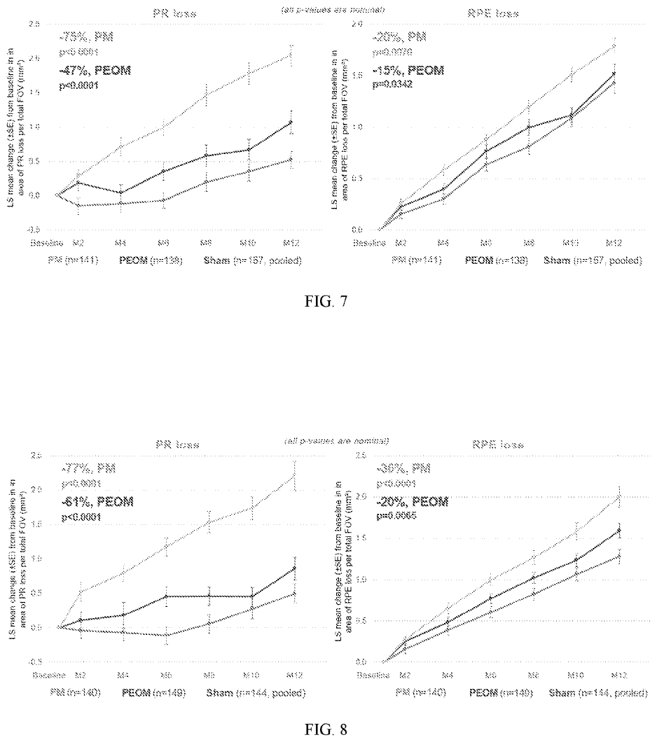

Spectral-domain optical coherence tomography (OCT) allows for cross sectional visualization of the neurosensory retina, including the photoreceptor (PR) layer, as well as the retinal pigment epithelium (RPE) and other retinal structures. Furthermore, OCT imaging permits analysis of outer retinal changes such as PR loss in areas outside of geographic atrophy lesions and has demonstrated that areas of PR loss in patients with GA often exceed the area of RPE atrophy. A post-hoc analysis that assessed the ability of pegcetacoplan treatment to reduce PR and RPE loss in patients with GA was performed using SD-OCT images acquired from a subgroup of patients in the OAKS and DERBY studies, namely those for whom Spectralis SD-OCT images were available. Automated, deep learning based segmentation of RPE loss and PR thickness on SD-OCT volumes acquired at baseline and months 2, 4, 6, 8, 10, and 12 was performed according to previously published methods. The difference in the change from baseline of RPE loss area and of PR loss area (determined based on PR thickness measurements) was compared among the monthly, every other month, and pooled sham treatment arms using LS Mean MMRM modeling. Results are presented in FIGS. 7 and 8. A statistically significant reduction in PR loss and RPE loss at 12 months was observed in the monthly and every other month pegcetacoplan treatment arms as compared with sham treatment in both DERBY and OAKS. The magnitude of the treatment effects at 12 months, as determined using LS mean MMRM modeling, are summarized below.

### DERBY

RPE loss
   EM vs. Sham Pooled: −36.0% difference (p<0.0001)
   Every other month vs. Sham pooled: −20.4% difference (p<0.0065)
   EM vs EOM: −19.6% difference (p=0.0103)·
PR loss
   EM vs. Sham Pooled: −77.4% difference (p<0.0001)
   Every other month vs. Sham pooled: −61.0% difference (p<0.0001)
   EM vs EOM: −42% difference (p=0.0785)

### OAKS

RPE loss
   EM vs. Sham Pooled: −19.7% difference (p=0.0070)
   Every other month vs. Sham pooled: −14.8% difference (p=0.0342)
   EM vs EOM: −5.7% difference (p=0.5282)
PR Loss
   EM vs. Sham Pooled: −74.5% difference (p<0.0001)
   EOM vs. Sham pooled: −47.7% difference (p<0.0001)
   EM vs EOM: −51.3% difference (p=0.0049)

### Safety

Pegcetacoplan was well tolerated in both Phase 3 studies. Table 2 shows the patient disposition and exposure at month 24 for the patients in the OAKS and DERBY studies.

TABLE 2
Patient disposition and exposure at Month 24

| | OAKS | | | DERBY | | |
|---|---|---|---|---|---|---|
| | PM | PEOM | Sham Pooled | PM | PEOM | Sham Pooled |
| ITT set | (N = 213) | (N = 212) | (N = 212) | (N = 206) | (N = 208) | (N = 207) |
| Completed study through | 144 (67.6%) | 169 (79.7%) | 158 (74.5%) | 147 (71.4%) | 161 (77.4%) | 161 (77.8%) |
| Month 24, n (%) | | | | | | |
| Discontinued study prior | 69 (32.4%) | 43 (20.3%) | 54 (25.5%) | 59 (28.6%) | 47 (22.6%) | 46 (22.2%) |
| to Month 24, n (%) | | | | | | |

19% (60/318) of study discontinuations were attributed to COVID-19

| | PM | PEOM | Sham Pooled | PM | PEOM | Sham Pooled |
|---|---|---|---|---|---|---|
| mITT set | (N = 202) | (N = 205) | (N = 207) | (N = 201) | (N = 201) | (N = 195) |
| Mean number of | 18.9 (6.08) | 10.2 (2.92) | 14.4 (6.50) | 18.7 (6.09) | 10.0 (2.80) | 14.7 (6.46) |
| injections/patient, n (SD) | | | | | | |
| Mean compliance, % | 87.4% | 90.5% | 87.9% | 85.6% | 89.0% | 88.7% |

Compliance % = injections administered/injections scheduled up to study completion or treatment discontinuation × 100
In Table 2, the ITT set included all randomized patients; mITT = modified ITT, defined as all randomized patients who received at least 1 injection of pegcetacoplan or sham and had baseline and at least 1 post-baseline value of geographic atrophy lesion area in the study eye; ITT = intent-to-treat; N = number of patients; PEOM = pegcetacoplan every other month; PM = pegcetacoplan monthly; SD = standard deviation.

Table 3 lists the overall TEAEs observed for the OAKS and DERBY studies over 24 months:

TABLE 3
TEAEs observed for OAKS and DERBY over 24 months

| | OAKS | | | | | | DERBY | | | | | |
|---|---|---|---|---|---|---|---|---|---|---|---|---|
| | PM | | PEOM | | Sham pooled | | PM | | PEOM | | Sham pooled | |
| | (N = 213) | | (N = 212) | | (N = 211) | | (N = 206) | | (N = 208) | | (N = 206) | |
| All TEAEs, n (%) | 192 | (90.1%) | 187 | (88.2%) | 175 | (82.9%) | 178 | (86.4%) | 180 | (86.5%) | 169 | (82.0%) |
| Ocular TEAEs in study eye, patients, n (%) | 133 | (62.4%) | 123 | (58.0%) | 98 | (46.4%) | 125 | (60.7%) | 108 | (51.9%) | 95 | (46.1%) |
| Non-ocular TEAEs, patients, n (%) | 174 | (81.7%) | 165 | (77.8%) | 154 | (73.0%) | 163 | (79.1%) | 142 | (68.3%) | 146 | (70.9%) |

| | | | | | | |
|---|---|---|---|---|---|---|
| Serious ocular TEAEs in the study eye, n (%) M | 5 (2.3%) 7 | 4 (1.9%) 4 | 1 (0.5%) 1 | 4 (1.9%) 4 | 2 (1.0%) 4 | 2 (1.0%) 2 |
| Endophthalmitis | 2 (0.9%) 2 | 3 (1.4%) 3 | 0 | 0 | 0 | 0 |
| Optic ischemic neuropathy | 2 (0.9%) 2 | 0 | 0 | 1 (0.5%) 1 | 0 | 0 |
| Retinal detachment | 1 (0.5%) 1 | 1 (0.5%) 1 | 0 | 0 | 0 | 0 |
| Uveitis | 0 | 0 | 0 | 0 | 2 (1.0%) 2 | 0 |
| Vitritis | 0 | 0 | 0 | 2 (1.0%) 2 | 0 | 0 |
| Visual acuity reduced | 0 | 0 | 1 (0.5%) 1 | 0 | 1 (0.5%) 1 | 0 |
| Papilledema | 1 (0.5%) 1 | 0 | 0 | 0 | 0 | 0 |
| Iridocyclitis | 0 | 0 | 0 | 0 | 1 (0.5%) 1 | 0 |
| Retinal tear | 0 | 0 | 0 | 1 (0.5%) 1 | 0 | 0 |
| Dry AMD | 0 | 0 | 0 | 0 | 0 | 1 (0.5%) 1 |
| Macular hole | 0 | 0 | 0 | 0 | 0 | 1 (0.5%) 1 |
| Hyphema | 1 (0.5%) 1 | 0 | 0 | 0 | 0 | 0 |

In Table 3, n indicates the number of patients; M indicates number of events; the events of endophthalmitis include infectious and non-infectious endophthalmitis; sham patients did not receive injections; AE = adverse event; AMD = age-related macular degeneration; N = number of patients; PEOM = pegcetacoplan every other month; PM = pegcetacoplan monthly; TEAE = treatment-emergent AE.

### Example 2—Post-Hoc Analysis of 24 Month Data

This Example describes a post-hoc analysis of results from the OAKS and DERBY studies described in Example 1. Functional data by lesion distance from foveal center were assessed over 24 months. Patients were divided into two subgroups: the first subgroup had a baseline lesion distance of ≥250 μm from the foveal center, and the second subgroup had a baseline lesion distance of <250 μm from the foveal center.

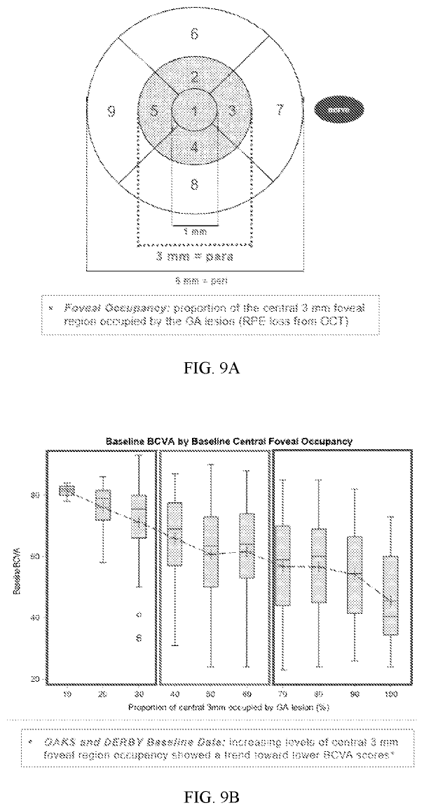

“Foveal Occupancy” was defined as proportion of the central 3 mm foveal region occupied by a GA lesion (see FIG. 9B). As shown in FIG. 9A, baseline BCVA from OAKS and DERBY patients was correlated with foveal occupancy, with increasing levels of foveal occupancy showing a trend toward lower BCVA scores.

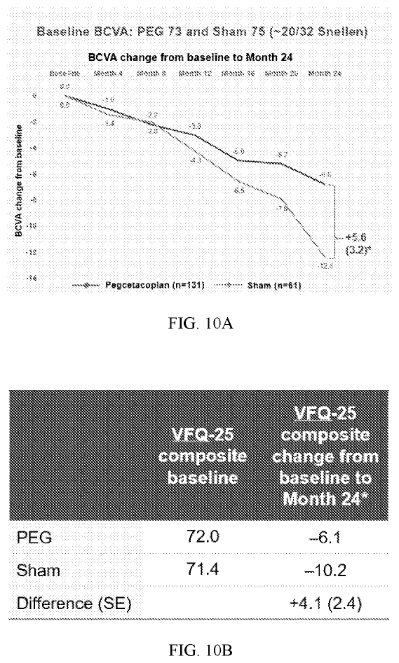

In the patient subgroup having lesions ≥250 μm away from the foveal center, pegcetacoplan slowed vision loss and improved quality of life in patients. As shown in FIG. 10A, patients treated with pegcetacoplan exhibited less of a BCVA change from baseline at 24 months, relative to sham-treated patients (pegcetacoplan treated patients had a baseline BCVA of 73, and sham treated patients had a baseline BCVA of 75, and patients treated monthly or every other month were combined). As shown in FIG. 10B, pegcetacoplan treated patients had better quality of life relative to sham treated patients at 24 months by over 4 points, assessed by VFQ-25. This subgroup included patients in the mITT sample with non-missing baseline measurements of RPE central 3 mm occupancy (C30) in regions 1-5 and lesion distance from fovea center. Mean changes were calculated among patients with BCVA measurements at each timepoint and thus samples varied across outcomes shown with no imputation for missing data. Adjusted differences from bootstrapped inverse probability weighted regressions controlling for baseline C30 in regions 1-5, study and fellow eye GA characteristics, study eye visual acuity, and patient characteristics.

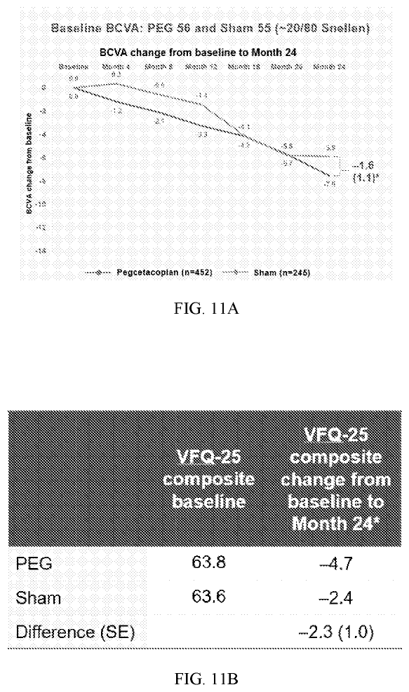

In the patient subgroup having lesions <250 μm from the foveal center, overall trends in BCVA (FIG. 11A) and VFQ-25 (FIG. 11B) changes over time were similar across pegcetacoplan and sham treated patients. This subgroup included patients in the mITT sample with non-missing baseline measurements of RPE central 3 mm occupancy (C3O) in regions 1-5 and lesion distance from fovea center. Mean changes were calculated among patients with BCVA measurements at each timepoint and thus samples varied across outcomes shown with no imputation for missing data. Adjusted differences from bootstrapped inverse probability weighted regressions controlling for baseline C30 in regions 1-5, study and fellow eye GA characteristics, study eye visual acuity, and patient characteristics.

### Example 3—Pegcetacoplan Treatment Prolongs Foveal Sensitivity in Patients with Geographic Atrophy (GA) Secondary to Age-Related Macular Degeneration (AMD)

This Example describes a post-hoc analysis of results from the OAKS study described in Example 1 that was performed in order to assess the effect of pegcetacoplan monthly (PM) or every other month (PEOM) vs sham treatment in delaying occurrence of foveal insensitivity measured by the 4 most centrally located stimulus points of the MAcular Integrity Assessment (MAIA) grid. Absolute scotomatous points of −1 dB were considered present when a reaction was not evoked by the brightest possible light stimulus.

Light insensitivity was defined as absence of response to −1 dB at the stimulus point. For the time-to-event analysis, a foveal light insensitivity event was defined as the first light insensitivity (−1 dB) occurring at all 4 central stimuli. Events were measured at the exact date of a patient visit and depicted via Kaplan-Meier plots. A Cox proportional hazards model was applied factoring treatment, choroidal neovascularisation in the fellow eye, baseline GA lesion area, and baseline number of central 4 scotomatous points (categorical). Hazard ratios (HRs) of PM and PEOM were estimated. Study eyes of patients with 4 central light insensitive points at baseline (floor effect) or who did not have a post-baseline microperimetry assessment were excluded from the analysis

At baseline, 18.3% of the 637 patients (PM, n=213; PEOM, n=212; sham pooled, n=212) enrolled in OAKS with a microperimetry assessment had 4 central scotomatous points (i.e., foveal light insensitivity) with mean NL-BCVA of 47.1 letters. Of these patients, 85.6% had baseline subfoveal involvement of the GA lesion. Additionally, 34.9% of patients with baseline microperimetry assessment had 2 or 3 central scotomatous points at baseline (mean NL-BCVA=56.8) and 46.8% had≤1 central scotomatous point at baseline (mean NL-BCVA=65.3). A total of 453 patients (PM, n=155; PEOM, n=152; sham pooled, n=146) who had≤3 central scotomatous points at baseline and a post-baseline microperimetry assessment were included in this analysis. Conversion of all 4 central points to scotoma in the study eye occurred in 54 (34.8%), 59 (38.8%), and 64 (43.8%) patients in the PM, PEOM, and sham pooled arms, respectively. PM significantly delayed the occurrence of foveal light insensitivity vs sham (HR: 0.66; 95% confidence interval [CI]: 0.46, 0.96; p=0.0282). PEOM similarly delayed the occurrence of foveal light insensitivity vs sham (HR: 0.64; 95% CI: 0.45, 0.92; p=0.0164).

Twenty-four-month data from the OAKS pivotal trial demonstrated a significant functional benefit of pegcetacoplan treatment in delaying onset of foveal light insensitivity in patients with GA reflected by the development of absolute scotomas at the 4 central stimulus points of the MAIA grid located closest to the foveal centre. Meaningful delays in loss of foveal light sensitivity were observed for both PM and PEOM regimens, supporting the use of pegcetacoplan treatment in maintaining foveal functionality of patients with GA lesions.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of maintaining, or reducing loss of, visual function of an eye of a subject suffering from or at risk of geographic atrophy (GA), e.g., GA secondary to age related macular degeneration (AMD), the method comprising intravitreally administering to an eye of the subject a composition comprising pegcetacoplan, wherein:
(a) the composition comprises about 15 mg pegcetacoplan; and
(b) wherein visual function of the eye of the subject is maintained, or loss of visual function is reduced, relative to control.

2. The method of claim 1, wherein visual function of the eye is assessed by microperimetry, monocular maximum reading speed, Functional Reading Independence (FRI) Index, best-corrected visual acuity (BCVA) (e.g., normal luminance BCVA (NL-BCVA) or low luminance BCVA (LL-BCVA)).

3. The method of claim 1 or 2, wherein after the administration, level of visual function of the eye is about 5%, 10%, 20%, 30%, 40%, 50%, or 60% better relative to a control.

4. The method of any one of claims 1-3, wherein visual function of the eye is assessed by microperimetry.

5. The method of claim 4, wherein after the administration, mean threshold sensitivity of the eye is about 0.25, about 0.5, about 0.75, or about 1 db higher, relative to a control.

6. The method of claim 4 or 5, wherein the eye of the subject has a geographic atrophy lesion, and microperimetry is assessed within an area peripheral to a lesion border.

7. The method of claim 6, wherein microperimetry is assessed within an area within about 500 microns (e.g., within about 400 microns, within about 300 microns, within about 250 microns, within about 200 microns, within about 150 microns) of each side of the lesion border.

8. The method of any one of claims 1-4, wherein after the administration the presence of foveal light sensitivity is prolonged relative to a control.

9. The method of claim 8, wherein the subject has a subfoveal GA lesion.

10. The method of claim 8, wherein the subject has reduced foveal light sensitivity relative to a control at the time treatment is started.

11. The method of any of claims 1-10, wherein the composition is administered monthly.

12. The method of any of claims 1-10, wherein the composition is administered every other month.

13. The method of any of claims 1-10, wherein the composition is administered by intravitreal injection between 6 and 12 times per year.

14. The method of any of claims 1-13 wherein the composition is administered in a volume of about 0.1 mL.

15. A method of decreasing new scotomatous points in an eye of a subject diagnosed with geographic atrophy, the method comprising administering to the eye of the subject a composition comprising about 15 mg pegcetacoplan for at least 24 months, and wherein the number of new scotomatous points is decreased relative to control.

16. The method of claim 15, wherein the pegcetocoplan is administered intravitreally.

17. The method of claim 15 or 16, wherein visual function of the eye is maintained, or loss of visual function is reduced, relative to control.

18. The method of any of claims 15-17, wherein visual function of the eye is assessed by microperimetry, monocular maximum reading speed, Functional Reading Independence (FRI) Index, best-corrected visual acuity (BCVA) (e.g., normal luminance BCVA (NL-BCVA) or low luminance BCVA (LL-BCVA)).

19. The method any of claims 15-18, wherein after the administration, level of visual function of the eye is about 5%, 10%, 20%, 30%, 40%, 50%, or 60% better relative to a control.

20. The method of any of claims 15-19, wherein visual function of the eye is assessed by microperimetry.

21. The method of any of claims 15-20, wherein the pegcetacoplan is administered in a volume of about 0.1 mL.

22. The method of any of claims 15-21, wherein the pegcetacoplan is administered monthly.

23. The method of any of claims 15-21, wherein the pegcetacoplan is administered every other month.

24. The method of any of any of claims 15-21, wherein the pegcetacoplan is administered between 6 and 12 times per year.

25. A method of improving the quality of life of a subject diagnosed with geographic atrophy, the method comprising administering a composition comprising about 15 mg pegcetacoplan to an eye of the subject, wherein said eye has a geographic atrophy lesion greater than or equal to 250 μm away from the foveal center, wherein the quality of life of the subject is improved relative to a control.

26. The method of claim 25, wherein the pegcetocoplan is administered intravitreally.

27. The method of claim 25 or 26, wherein visual fuction of the at least one eye is maintained, or loss of visual function is reduced, relative to control.

28. The method of any of claims 25-27, wherein visual function of the eye is assessed by microperimetry, monocular maximum reading speed, Functional Reading Independence (FRI) Index, best-corrected visual acuity (BCVA) (e.g., normal luminance BCVA (NL-BCVA) or low luminance BCVA (LL-BCVA)).

29. The method of any of claims 25-28, wherein after the administration, level of visual function of the eye is about 5%, 10%, 20%, 30%, 40%, 50%, or 60% better relative to a control.

30. The method of any of claims 25-29, wherein visual function of the eye is assessed by microperimetry.

31. The method of any of claims 25-30, wherein the pegcetacoplan is administered in a volume of about 0.1 mL.

32. The method of any of claims 25-31, wherein the pegcetacoplan is administered monthly.

33. The method of method of any of claims 25-31, wherein the pegcetacoplan is administered every other month.

34. The method of any of claims 25-31, wherein the pegcetacoplan is administered between 6 and 12 times per year.

35. The method of any of claims 25-31, the method comprising determining that the subject has a geographic atrophy lesion greater than or equal to 250 μm away from the foveal center of at least one eye and administering pegcetacoplan to that eye.
